(19) United States
(12) Patent Application Publication
(10) Pub. No.: US 2025/0352827 A1
(43) Pub. Date: Nov. 20, 2025

(54) ARC THERAPY APPARATUSES, METHODS FOR OPERATING THE SAME, AND MAGNETIC FIELD ADJUSTMENT DEVICES

(71) Applicant: MEVION MEDICAL EQUIPMENT CO., LTD., Suzhou (CN)

(72) Inventors: Zhihong ZHENG, Suzhou (CN); Bo WU, Suzhou (CN); Jinwen SHUAI, Suzhou (CN); Xuemin BAI, Suzhou (CN)

(73) Assignee: MEVION MEDICAL EQUIPMENT CO., LTD., Suzhou (CN)

(21) Appl. No.: 19/287,775

(22) Filed: Jul. 31, 2025

Related U.S. Application Data
(63) Continuation-in-part of application No. PCT/CN2023/133260, filed on Nov. 22, 2023.

(30) Foreign Application Priority Data
Aug. 28, 2023 (CN) 202311087319.8

(51) Int. Cl. A61N 5/10; A61N 5/00

(52) U.S. Cl. CPC A61N 5/1081; A61N 5/1039; A61N 5/1067; A61N 5/107; A61N 2005/005; A61N 2005/1074

(57) ABSTRACT

Provided are an arc therapy apparatus, a method for operating the arc therapy apparatus, and a magnetic field adjustment device. The arc therapy apparatus includes a particle accelerator configured to generate and adjust a particle beam, a beam delivery system configured to deliver the particle beam, a subject positioning system configured to position a subject, and a treatment gantry configured to drive the particle accelerator and the beam delivery system to rotate around an isocenter to perform arc therapy. The magnetic field adjustment device is applied to the particle accelerator mounted on the treatment gantry of the arc therapy apparatus. The magnetic field adjustment device is configured to adjust a particle deflection magnetic field based on a change of a gantry angle of the treatment gantry, such that the particle beam generated by a particle radiation source satisfies a preset particle beam condition under the particle deflection magnetic field.

### CROSS-REFERENCE TO RELATED APPLICATIONS

This application is a continuation-in-part of International Patent Application No. PCT/CN2023/133260, filed on Nov. 22, 2023, which claims priority to Chinese Patent Application 202311087319.8, filed on Aug. 28, 2023, the entire contents of each of which are incorporated herein by reference.

### TECHNICAL FIELD

The present disclosure relates to the field of radiation therapy, and in particular, relates to arc therapy apparatuses, methods for operating the same, and magnetic field adjustment devices.

### BACKGROUND

Particle radiotherapy is a modern tumor treatment approach that utilizes high-energy particles to precisely target tumor cells, delivering energy accurately within the tumor cells to destroy them. Particle radiotherapy enables precise control of a dose and a range of radiation, thereby reducing damage to healthy tissues while improving treatment efficacy.

Particle Arc Therapy (PAT) is a particle radiotherapy approach in which a treatment gantry continuously rotates to adjust an irradiation angle during the treatment process. A Treatment Planning System (TPS) for PAT distributes the uncertainty of particle range across various angles, thus achieving strong robustness.

During the rotation of the treatment gantry, coils used to provide a particle deflection magnetic field may have an offset, causing deflection in a direction of the magnetic field, ultimately affecting the stability of particle beam-related parameters.

Therefore, it is desirable to provide an arc therapy apparatus, a method for operating the arc therapy apparatus, and a magnetic field adjustment device to ensure that variations in the particle beam-related parameters remain within an acceptable range during the rotation of the treatment gantry.

### SUMMARY

One or more embodiments of the present disclosure provide a magnetic field adjustment device, which is applied to a particle accelerator mounted on a treatment gantry of an arc therapy apparatus. The particle accelerator rotates along with the treatment gantry during rotation of the treatment gantry. The particle accelerator includes a magnetic field providing device configured to provide a particle deflection magnetic field. The magnetic field adjustment device is configured to adjust the particle deflection magnetic field based on a change of a gantry angle of the treatment gantry, such that a particle beam generated by a particle radiation source satisfies a preset particle beam condition under the particle deflection magnetic field. The preset particle beam condition indicates a preset range of each of one or more of an energy level, a beam spot size, and a beam spot position of the particle beam.

One or more embodiments of the present disclosure provide an arc therapy apparatus. The arc therapy apparatus includes a particle accelerator configured to generate and adjust a particle beam, a beam delivery system configured to deliver the particle beam, a subject positioning system configured to position a subject, and a treatment gantry configured to drive the particle accelerator and the beam delivery system to rotate around an isocenter to perform arc therapy.

One or more embodiments of the present disclosure provide a method for operating an arc therapy apparatus. The method includes: acquiring a treatment plan, the treatment plan including a plurality of preset angular intervals and a preset dose of a particle beam corresponding to each of the preset angular intervals; and rotating a treatment gantry of the arc therapy apparatus to each of the preset angular intervals, respectively, to complete an irradiation process of the particle beam corresponding to each of the preset angular intervals.

### BRIEF DESCRIPTION OF THE DRAWINGS

The present disclosure is further illustrated by way of exemplary embodiments, which is described in detail through the accompanying drawings. These embodiments are not limiting, and in these embodiments, the same numbering denotes the same structure, wherein:

FIG. 1 is a structural block diagram of an arc therapy apparatus according to some embodiments of the present disclosure.

FIG. 2 is a schematic diagram of a portion of a three-dimensional structure of an arc therapy apparatus according to some embodiments of the present disclosure.

FIG. 3 is a structural block diagram of a particle accelerator according to some embodiments of the present disclosure.

FIG. 4 is a schematic diagram of an exemplary structure of a beam spot size adjustment device according to some embodiments of the present disclosure.

FIG. 5 is a schematic diagram of an exemplary structure of a shielding plate according to some embodiments of the present disclosure.

FIG. 6 is a flowchart of an exemplary process of a method for operating an arc therapy apparatus according to some embodiments of the present disclosure.

FIG. 7 is a flowchart of an exemplary process for adjusting a treatment couch according to some embodiments of the present disclosure.

FIG. 8 is a flowchart of an exemplary process of a first implementation manner for achieving arc therapy according to some embodiments of the present disclosure.

FIG. 9 is a flowchart of an exemplary process of a second implementation manner for achieving arc therapy according to some embodiments of the present disclosure.

FIG. 10 is a structural block diagram of an operating device according to some embodiments of the present disclosure.

FIG. 11 is a structural block diagram of a magnetic field adjustment device according to some embodiments of the present disclosure.

FIG. 12 is a three-dimensional view of a magnetic field providing device and execution components according to some embodiments of the present disclosure.

FIG. 13 is a side view of a magnetic field providing device and execution components according to some embodiments of the present disclosure.

FIG. 14 is a top view of a magnetic field providing device and execution components according to some embodiments of the present disclosure.

FIG. 15 is a structural block diagram of an execution component according to some embodiments of the present disclosure.

FIG. 16 is a schematic diagram of an exemplary structure of an execution component according to some embodiments of the present disclosure.

FIG. 17 is a control flowchart of a magnetic field adjustment device according to some embodiments of the present disclosure.

FIG. 18 is a schematic diagram of an exemplary structure of a computer program product according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments are readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. As used in the claims and the specification, the term “and/or” is merely used to describe an associative relationship between related objects, indicating that three possible relationships may exist. For example, “A and/or B” may represent the following three scenarios: A alone, both A and B together, or B alone. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It may be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

It may be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

These and other features, and characteristics of the present disclosure, as well as the methods of operation and functions of the related elements of structure and the combination of parts and economics of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

The present disclosure provides an apparatus (i.e., an arc therapy apparatus) that can provide particle arc radiation therapy. By increasing an irradiation angle, the arc therapy apparatus can reduce an integral dose of a high-energy particle beam outside a target region. Moreover, since a treatment gantry of the arc therapy apparatus can continuously deliver irradiation during rotation (or quickly resume irradiation after rotating to a preset angle), the overall treatment time remains unaffected.

Some embodiments of the present disclosure provide an arc therapy apparatus (hereinafter referred to as the apparatus).

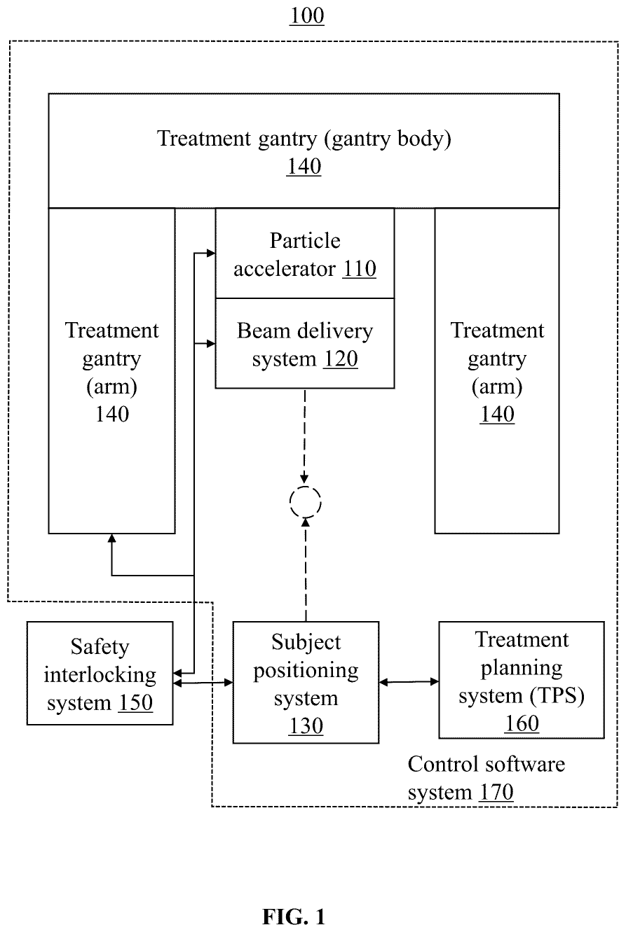

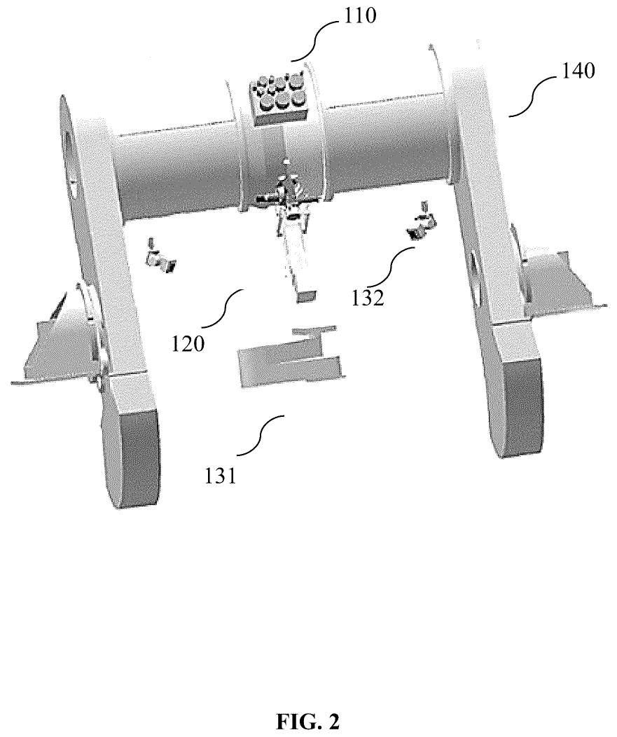

FIG. 1 is a structural block diagram of an arc therapy apparatus according to some embodiments of the present disclosure. FIG. 2 is a schematic diagram of a portion of a three-dimensional structure of an arc therapy apparatus according to some embodiments of the present disclosure.

In some embodiments, an arc therapy apparatus 100 comprises a particle accelerator 110, a beam delivery system 120, a subject positioning system 130, and a treatment gantry 140. The particle accelerator 110 is configured to generate and adjust a particle beam. The beam delivery system 120 is configured to deliver the particle beam. The subject positioning system 130 is configured to position a subject (e.g., a patient). The treatment gantry 140 is configured to drive the particle accelerator 110 and the beam delivery system 130 to rotate around an isocenter to perform arc therapy.

The particle accelerator 110 is a device configured to accelerate charged particles (e.g., protons or heavy ions) to high energies.

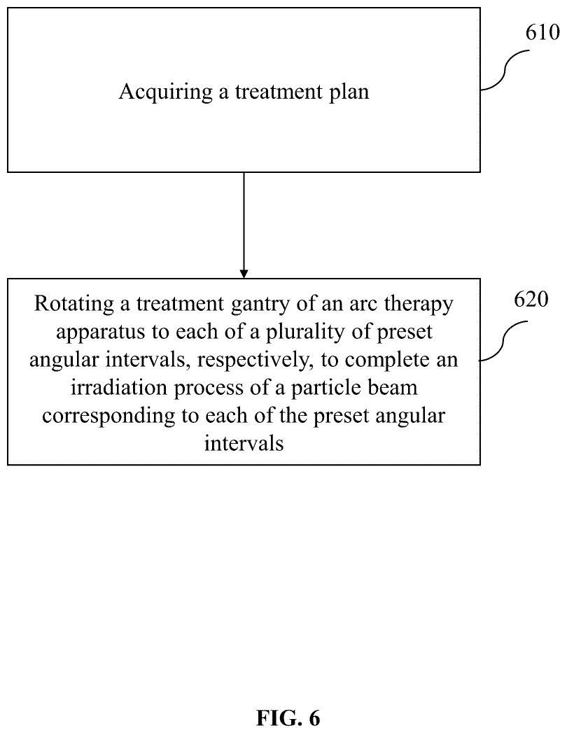

In some embodiments, the particle accelerator 110 may generate the particle beam based on a treatment plan. By adjusting an energy and a beam spot size of the particle beam, precise irradiation of a tumor tissue can be achieved. The particle beam may be a high-energy proton beam or a heavy ion beam (e.g., a helium ion beam, a carbon ion beam, etc.). More descriptions regarding the treatment plan may be found in the related descriptions of FIG. 6.

In some embodiments, the particle accelerator 110 may include at least one of an isochronous cyclotron, a synchrocyclotron, or the like.

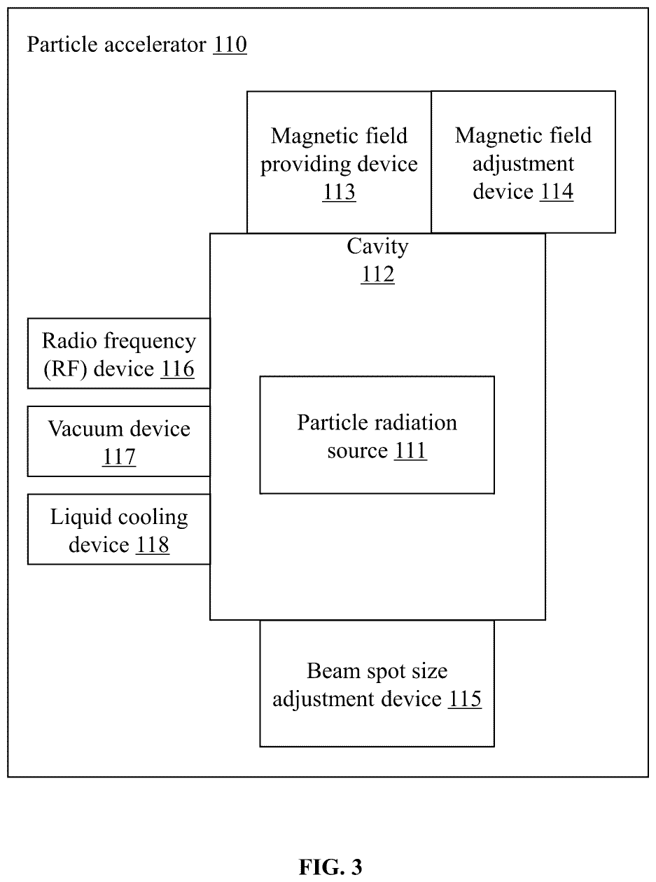

In some embodiments, the particle accelerator 110 may include a particle radiation source 111, a cavity 112, a magnetic field providing device 113, a magnetic field adjustment device 114, a beam spot size adjustment device 115, a radio frequency (RF) device 116, a vacuum device 117, and a liquid cooling device 118. For further details regarding this section, refer to the related descriptions of FIG. 3.

The beam delivery system 120 is configured to transport the particle beam from the particle accelerator 110 to a target region (e.g., a lesion region) of the subject, ensuring accurate delivery of a preset dose of the particle beam as defined in the treatment plan. In some embodiments, the beam delivery system 120 precisely transport the particle beam from the particle accelerator 110 to a treatment position by magnetic field control, ensuring accurate positioning and transmission of the particle beam.

The subject positioning system 130 is configured to achieve correct positioning of the subject. The correct positioning refers to a position and a posture of the subject when the particle beam can be accurately irradiated to the target region in the subject. With the correct positioning, the particle beam can be strictly aligned with the target region, minimizing the impact on normal tissues around the target region.

In some embodiments, as shown in FIG. 2, the beam delivery system 120 includes a treatment head (not shown in FIGS. 1, 2); and/or the subject positioning system 130 includes a treatment couch 131 and an imaging system 132.

The treatment head refers to a core component located at an end of the beam delivery system 120, which is used for final shaping of the particle beam generated by the accelerator, energy regulation, and dosage control to ensure that the radiation accurately acts on the target region.

In some embodiments, the treatment head includes an active beam scanning treatment head and/or a passive scattering treatment head.

In some embodiments, the active beam scanning treatment head may include a scanning magnet, an ionization chamber, a range shifter, an adaptive aperture, or the like. The scanning magnet is configured to focus and deflect the particle beam. The ionization chamber is configured to provide real-time feedback on the energy and energy distribution of the particle beam stream. The range shifter is configured to adjust a penetration depth of the particle beam (e.g., adjusting a spread-out Bragg peak (SOBP) of the particle beam). The adaptive aperture may be adaptively self-adjusted based on a shape and a size of the target region (e.g., a tumor region, etc.), so that a shape and a size of the particle beam can be matched with the tumor morphology. The adaptive irradiation approach can better conform to irregularly shaped tumors, thereby enhancing the personalization and precision of the treatment plan. A combination of components including the scanning magnet, the ionization chamber, the range shifter, and the adaptive aperture enables flexible treatment modalities.

The active beam scanning treatment head allows for real-time adjustments and optimization of the treatment plan based on actual conditions and disease progression of the subject to better meet the subject's treatment requirements. Due to its high efficiency and precision, the active beam scanning treatment head can reduce demands on the particle accelerator, thus improving the stability and reliability of the apparatus.

The active beam scanning treatment head enables rapid scanning and adjustment of the particle beam, resulting in a more efficient treatment process. Compared to the passive scattering treatment head, the active beam scanning treatment head offers a faster scanning speed and a shorter irradiation duration, which reduces a treatment duration for the subject and alleviates discomfort and anxiety. Additionally, the active beam scanning treatment head is technologically more advanced and sophisticated, and its design can reduce system complexity while improving the stability and reliability of the apparatus.

The passive scattering treatment head is configured to spread and disperse the particle beam uniformly so that the distribution of the particle beam better matches the morphology of the target region for radiation therapy. For example, the passive scattering treatment head may convert a high-energy proton beam into a wider and more dispersed beam spot, so that the distribution of the particle beam can be adapted to the shape and the size of the target region, which ensure accurate coverage of the entire tumor region during treatment, thereby improving treatment precision and efficacy.

In some embodiments of the present disclosure, the passive scattering treatment head may include a double scattering treatment head, a single scattering treatment head, or the like.

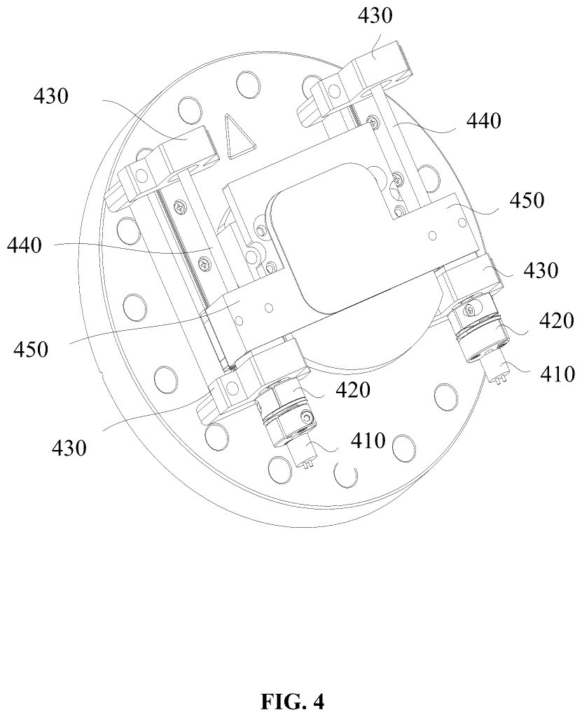

It should be noted that scattering may cause the energy of the particle beam around the target region to exceed a required therapeutic energy, posing a risk of damage to healthy tissues. Therefore, the passive scattering treatment head may be used in conjunction with a blocking mechanism. By adjusting a position of the blocking mechanism, the shape and the distribution of the particle beam can be further controlled to achieve more precise irradiation. More description about the blocking mechanism may be found in the related descriptions of FIG. 4.

Compared with the active beam scanning treatment head, the passive scattering treatment head is technically simpler and does not require complex equipment such as the scanning magnet, which not only reduces the requirements for the particle accelerator but also lowers equipment and maintenance costs.

In some embodiments, the treatment couch 131 may be electrically or mechanically driven, and may perform translational, rotational, and vertical movements along a plurality of axes to achieve position adjustments. In some embodiments, a degree of freedom (DOF) of the treatment couch 131 may be set based on actual requirements. For example, the DOF of the treatment couch may be one of 3, 4, 5, 6, or the like.

The imaging system 132 is a device configured to acquire intraoperative medical imaging data of the subject. In some embodiments, the imaging system 132 may include at least one of an X-ray device, a Computed Tomography (CT) scanner, a Magnetic Resonance (MR) scanning device, or the like.

In some embodiments of the present disclosure, the treatment head ensures precise irradiation of the target region while minimizing damage to healthy tissues, thereby improving treatment accuracy. The flexibility of the beam delivery system combined with the precise positioning capability of the subject positioning system enables the treatment plan to be more personalized, achieving the correct positioning based on individual differences between subjects and the location of the target region, which lays the foundation for subsequent precision treatment. The imaging system allows technical personnel to monitor the position, the posture, and anatomical changes of the subject in real time before and during treatment, thereby ensuring correct delivery of the particle beam and maintaining treatment accuracy and safety.

In some embodiments, the particle accelerator 110 and the beam delivery system 120 are mounted on the treatment gantry 140.

The treatment gantry 140 is a rotating component in the arc therapy apparatus 100. By way of example, as shown in FIG. 1, the treatment gantry 140 includes two gantry arms and a gantry body between the arms. The two gantry arms are respectively pivotally connected to a fixed base, and the particle accelerator is mounted on the gantry body.

In some embodiments, the treatment gantry 140 may drive the particle accelerator 110 and the beam delivery system 120 to rotate around the isocenter, so that the particle beam can irradiate the target region at different angles and with different energy levels and beam spot sizes, realizing omni-directional arc therapy. Theoretically, throughout a full angular range of an operation of the arc therapy apparatus, extension lines of a rotation axis of the treatment gantry 140, a rotation axis of the treatment head, and a rotation axis of the treatment couch should intersect at a point (e.g., a point inside the subject), which is referred to as the isocenter (ISO) or an isocenter point.

In some embodiments, the treatment gantry 140 is configured with an arc-shaped track rotatable around the isocenter, as illustrated in FIG. 2. A starting point and an ending point of the arc-shaped track are defined as an upper point and a lower point of the subject in a lying position, respectively.

In some embodiments, the arc-shaped track is in an O-XYZ three-dimensional coordinate system, where an origin point O of the coordinate system coincides with the isocenter. The rotation axis of the treatment gantry (i.e., the central axis of the arc-shaped track) is an X-axis of the coordinate system, a vertical direction from a ground toward the treatment couch is defined as a positive direction of a Z-axis of the coordinate system, and an Y-axis of the coordinate system may be any axis perpendicular to both the X-axis and the Z-axis, with an XOY plane parallel to the ground. A rotation angle of the treatment gantry 140 is defined as an angle, within the ZOY plane, between the positive Z-axis and a line connecting the particle accelerator 110 and/or the beam delivery system 120 with the origin point O. When the subject is in the lying position on the treatment couch, the subject is considered to be in the horizontal direction within the XOY plane.

For example, when the line connecting the particle accelerator 110 and/or the beam delivery system 120 with the origin point O is vertical to the ground, and the particle accelerator 110 and/or the beam delivery system 120 is located directly above the origin point O, the rotation angle of the treatment gantry 140 is 0°. When the line connecting the particle accelerator 110 and/or the beam delivery system 120 with the origin point O is parallel to the ground and the particle accelerator 110 and/or the beam delivery system 120 is located in a negative direction of the Y-axis, the rotation angle of the treatment gantry 140 is −90°.

In some embodiments, the arc-shaped track may drive the particle accelerator 110 and the beam delivery system 120 to rotate within a preset angular range. The preset angular range is a range that covers all clinical treatment angles, which may be set by those in the art based on experience. For example, the preset angular range may be −5° to 185°.

In some embodiments of the present disclosure, highly flexible rotational motion can be achieved by the treatment gantry, allowing the particle beam to rotate in a range of 190°, covering a wide range of treatment angles, which facilitates multi-angle precise irradiation tailored to different tumor locations and shapes, thereby improving treatment efficacy.

The movement of the treatment gantry combined with the rotational ability of the treatment couch allows for precise subject positioning. By ensuring the accurate alignment of the particle beam with the target region, radiation damage to the surrounding normal tissues can be minimized to improve treatment accuracy and safety. The particle accelerator is mounted on the treatment gantry and rotates along with the treatment gantry. This design reduces apparatus complexity by eliminating the need for beam transport lines, thereby simplifying the overall structure. The rotation of the particle accelerator with the treatment gantry also improves beam stability, as beam transport lines inherently introduce instability factors. Since the apparatus does not require beam transport lines, maintenance costs and failure rates are reduced, enhancing stability and reliability of the apparatus. The elimination of instability sources ensures smoother beam motion, contributing to the consistency of the particle beam and guaranteeing precise irradiation.

In addition, the rotation of the particle accelerator with the treatment gantry increases the flexibility and applicability of radiotherapy. By rotating the gantry, irradiation from different angles and directions can be achieved, making the treatment more comprehensive and efficient.

In some embodiments, as shown in FIG. 1, the arc therapy apparatus 100 further comprises a safety interlocking system 150, and/or a treatment planning system (TPS) 160, and/or a control software system 170. The safety interlocking system 150 may be configured to perform safety monitoring on the particle accelerator 110, the beam delivery system 120, the subject positioning system 130, and the treatment gantry 140. The TPS 160 may be configured to generate a treatment plan based on preoperative medical imaging data of the subject. The control software system 170 may be configured to validate the treatment plan and record treatment process data.

The safety interlocking system 150 may perform safety monitoring and control on components such as the particle accelerator 110, the subject positioning system 130, and the treatment gantry 140.

In some embodiments, the safety interlocking system 150 may also include a radiation safety control subsystem to ensure that the radiation dose is within a safe range to prevent overexposure to the subject and an operator.

The TPS 160 may be a medical software used to plan, optimize, and simulate a radiation treatment protocol. For example, the TPS 160 may analyze the preoperative medical imaging data of the subject, convert the preoperative medical imaging data into a three-dimensional image model to assist a physician determine a location, a size, and a shape of a tumor, and a structure of tissues surrounding the tumor, and generate a plurality of treatment protocols for simulation and optimization. The treatment protocol with the best therapeutic effect and minimal damage to healthy tissues is then selected as the treatment plan.

The control software system 170 is a core control part of the arc therapy apparatus 100. The control software system 170 may provide the user with a system interface to receive and manage the treatment plan, and record the treatment process data (e.g., the intraoperative medical imaging data, etc.), control the treatment process, and validate the accuracy of the treatment plan.

In some embodiments of the present disclosure, the safety interlocking system, the treatment planning system, and the control software system collectively play critical and interrelated roles in the arc therapy apparatus. The integration of the safety interlock system and control software system ensures safety and precision during treatment, preventing accidents and safeguarding the health of the subject and the operator. The treatment planning system enables the generation of precise treatment plans tailored to the subject's anatomical structure, maximizing therapeutic efficacy for physician reference. Meanwhile, the control software system manages the operation and control of the entire arc therapy apparatus, ensuring efficient, accurate, and safe treatment delivery while providing comprehensive support and safeguards for arc therapy. The effective coordination of these systems guarantees the successful implementation of the arc therapy and an optimal treatment experience for the subject.

FIG. 3 is a structural block diagram of a particle accelerator according to some embodiments of the present disclosure.

In some embodiments, as shown in FIG. 3, the particle accelerator 110 includes a particle radiation source 111, a cavity 112, and a magnetic field providing device 113. The particle radiation source 111 is configured to generate a particle beam, and particles of the particle beam are protons or heavy ions. The cavity 112 is configured to allow the particle beam to be accelerated within the cavity. The magnetic field providing device 113 is configured to provide a particle deflection magnetic field to the cavity. As the particle accelerator 110 rotates following the treatment gantry 140, components (including the particle radiation source 111, the cavity 112, and the magnetic field providing device 113) of the particle accelerator rotate along with the treatment gantry 140.

The particle radiation source 111 is a source for generating the particle beam, such as a component for generating high-energy charged particles.

In some embodiments, the particle radiation source 111 may include at least one of an electron beam ion source, a high charge state Electron Cyclotron Resonance (ECR) source, a radio frequency (RF) ion source, or the like.

The cavity 112 refers to a space in the particle accelerator 110 where the particle beam is accelerated. As the particle beam passes through the cavity, it gains further acceleration to reach a required energy level for the arc therapy. The cavity 112 may include one of a resonance cavity, an RF resonance cavity, or the like.

In some embodiments, the magnetic field providing device 113 may be mounted on the cavity 112.

The magnetic field providing device 113 refers to a device or component that provides a particle deflection magnetic field to the cavity 112. When the particle beam propagates in the particle accelerator 110, the magnetic field providing device 113 deflect the particle beam to maintain a trajectory of the particle beam.

In some embodiments, the magnetic field providing device 113 may include one or more superconducting coils.

In the arc therapy apparatus, the superconducting coil(s) are used to generate a high-intensity magnetic field for deflecting and controlling the propagation of the particle beam. As an example, liquid helium may be used to create a cryogenic environment (e.g., 4 Kelvin, near absolute zero).

The embodiments of the present disclosure do not limit the material of the superconducting coil(s). The material of the superconducting coil(s) may include a metal material or an alloy material. For example, the material of the superconducting coil(s) may include a copper oxide, a niobium-titanium alloy, a niobium-tin alloy, or the like.

The embodiments of the present disclosure do not limit the count of the superconducting coil(s). For example, the count of the superconducting coil(s) may be 1, 2, 3, 4, 10, or the like.

In some embodiments, the particle accelerator 110 further includes a magnetic field adjustment device 114. The magnetic field adjustment device 114 is an apparatus or component for adjusting the particle deflection magnetic field.

The magnetic field adjustment device 114 is disposed outside the cavity 112 and is configured to adjust a direction and a strength of the particle deflection magnetic field. In some embodiments, the magnetic field adjustment device 114 may be configured as various structures. For example, the magnetic field adjustment device 114 may be a movement mechanism that adjusts a position and/or an orientation of the magnetic field providing device 113. As another example, the magnetic field adjustment device 114 may be one or more magnets that adjust the direction and the strength of the particle deflection magnetic field.

In some embodiments, the particle deflection magnetic field may be adjusted in a variety of ways. For example, the control software system 170 may adjust the direction and the strength of the particle deflection magnetic field by adjusting a direction and a magnitude of a current in the superconducting coil(s). As another example, the direction and the strength of the particle deflection magnetic field may also be adjusted by the magnetic field adjustment device 114.

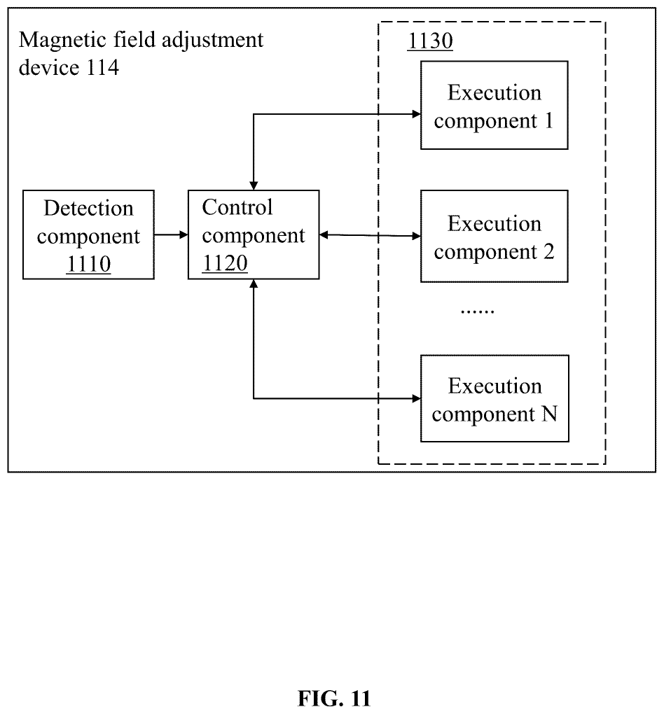

More descriptions regarding the magnetic field adjustment device and how to adjust the direction and the strength of the particle deflection magnetic field may be found in the related descriptions of FIGS. 11 to 17.

In some embodiments of the present disclosure, the magnetic field adjustment device enables precise adjustment and control of the particle deflection magnetic field. This ensures accurate adjustment of the trajectory and parameters of the particle beam, thereby enhancing the precision and efficacy of radiation therapy. The particle radiation source generates a high-energy particle beam capable of deeper tissue penetration, allowing more effective targeting of tumor cells while minimizing damage to healthy tissues. Due to the particle beam's precise controllability and high-energy characteristics, the particle accelerator delivers more accurate and higher-quality radiation therapy, helping the subject achieve optimal treatment outcomes.

In some embodiments, as shown in FIG. 3, the particle accelerator 110 further includes a beam spot size adjustment device 115 configured to adjust a beam spot size of the particle beam.

The beam spot size refers to a full width at half maximum (FWHM) of the dose distribution profile along a propagation direction of the particle beam.

In some embodiments, the beam spot size adjustment device 115 may adjust the beam spot size of the particle beam to accommodate different treatment requirements, thereby enhancing the flexibility and personalization of radiotherapy. For example, when treating tumors of varying sizes, the beam spot size may be adjusted to match different tumor dimensions and shapes. The beam spot size adjustment device 115 may adjust the beam spot size under different settings. For example, at a minimum setting of the beam spot size adjustment device 115, the beam spot size may be adjusted to a diameter of 1 cm, suitable for relatively small tumors, while at a maximum setting of the beam spot size adjustment device 115, the beam spot size may be adjusted to a diameter of 1.5 cm, suitable for relatively large tumors.

Different subjects or tumor locations may require varying beam spot sizes to achieve optimal therapeutic outcomes. By incorporating the beam spot size adjustment device, physicians can dynamically tailor the beam spot size based on specific clinical needs, enabling personalized treatment planning and improving treatment precision and efficacy. Adjusting the beam spot size allows better conformity to the tumor's shape and dimensions, ensuring more accurate coverage of the tumor region while minimizing radiation exposure to surrounding healthy tissues. This enhances both the accuracy and safety of radiotherapy, providing optimal protection to adjacent normal structures. Furthermore, proper beam spot size adjustment facilitates improved dose distribution across different depths and volumes, ensuring comprehensive tumor coverage and delivering sufficient therapeutic dose to cancer cells for enhanced treatment effectiveness. The beam spot size adjustment device significantly increases the flexibility of radiotherapy. Physicians can adapt the beam spot size at various treatment stages and angles according to real-time needs, addressing potential variations during therapy and enabling more precise and effective treatment delivery.

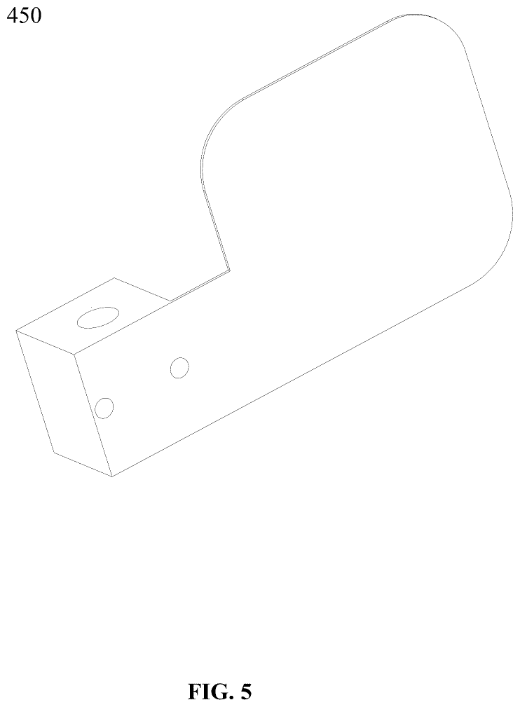

FIG. 4 is a schematic diagram of an exemplary structure of a beam spot size adjustment device according to some embodiments of the present disclosure. FIG. 5 is a schematic diagram of an exemplary structure of a shielding plate according to some embodiments of the present disclosure.

In some embodiments, as shown in FIGS. 4 and 5, the beam spot size adjustment device 115 includes at least one adjustment component 1151. Each of the at least one adjustment component 1151 includes a second drive controller (not shown in FIG. 4), a second drive mechanism 410, a second transmission mechanism 430, and a blocking mechanism 450. In each of the at least one adjustment component 1151, the second transmission mechanism 430 is fixedly connected to the blocking mechanism 450, and the second drive controller is configured to drive the second drive mechanism 410 to move based on a second control instruction corresponding to the adjustment component 1151, such that the second drive mechanism 410 drives the second transmission mechanism 430 to move, to adjust a blocking state of the blocking mechanism 450.

The adjustment component 1151 is configured to adjust the beam spot size of the particle beam. For example, the adjustment component may adjust the beam spot size of the particle beam by adjusting the scattering of the particle beam. A count of the at least one adjustment component 1151 may be set by a person skilled in the art based on experience.

The second drive controller is a controller for controlling other components in the beam spot sizing device 115. In some embodiments, the second drive controller is configured to receive the second control instruction and convert the second control instruction into a control signal for the second drive mechanism.

The second control instruction is configured to increase and/or decrease the beam spot size of the particle beam, which may be input by a physician. The control signal is a signal to adjust a movement of the second drive mechanism 410.

The second drive mechanism 410 is a mechanical structure in the beam spot size adjustment device 115 for driving the second transmission mechanism 430. In some embodiments, the second drive mechanism 410 is configured to receive the control signal from the second drive controller, drive the second transmission mechanism 430 to move, to drive a movement of the blocking mechanism 450 to adjust the beam spot size.

The second transmission mechanism 430 refers to a transmission structure in the beam spot size adjustment device 115. In some embodiments, the second transmission mechanism 430 may be fixedly connected to the blocking mechanism 450 for transmitting the motion of the second drive mechanism 410 to adjust the blocking state of the blocking mechanism 450.

The blocking mechanism 450 is configured to adjust the beam spot size. Through adjustments of the blocking mechanism 450, the particle beam may be scattered or not scattered, thereby changing the beam spot size.

In some embodiments, when a plurality of adjustment components are provided, a plurality of blocking mechanisms 450 may be arranged circumferentially around the particle beam. As an example, when two blocking mechanisms 450 are provided, the two blocking mechanisms 450 may be positioned in mirror symmetry about a central axis of the particle beam.

In some embodiments, the blocking state of the blocking mechanism 450 may include an open state and a closed state. When the second transmission mechanism 430 moves in a second direction, the blocking mechanism 450 is in the open state. In the open state, the scattering of the particle beam is restricted or altered, and the beam spot size of the particle beam decreases. When the second transmission mechanism 430 moves in a first direction, the blocking mechanism 450 is in the closed state. In the closed state, the restriction on the scattering of the particle beam is removed, and the beam spot size of the particle beam increases. The first direction refers to a movement direction of the second transmission mechanism 430 when the beam spot size increases. The second direction refers to a movement direction of the second transmission mechanism 430 when the beam spot size decreases.

In some embodiments of the present disclosure, the beam spot size can be flexibly adjusted through the second drive controller in the adjustment component. The beam spot size adjustment device enables real-time adjustment of the beam spot size during treatment according to the size and the position of the tumor, which helps optimize the treatment plan and ensure adequate coverage of the target region by the particle beam, thereby improving therapeutic outcomes. By precisely controlling the beam spot size, radiation damage to surrounding healthy tissues can be reduced and unnecessary irradiation of normal tissues can be avoided, thereby improving the safety and tolerability of the treatment. Adjusting the beam spot size also allows for better conformity to the shape and the size of the tumor, enabling more efficient utilization of the particle beam energy and enhancing treatment efficacy.

In some embodiments, the second drive mechanism 410 include a motor; and/or the second transmission mechanism 430 includes a lead screw; and/or the blocking mechanism 450 includes a plurality of shielding plates arranged in pairs, each pair of the shielding plates being symmetrically arranged along a central line of the blocking mechanism.

In some embodiments, the motor may be one of a servo motor, a stepper motor, or the like.

In some embodiments, a surface of the lead screw is provided with threads, and the lead screw may cooperate with a nut to convert rotational or linear motion of the motor to linear motion of the lead screw. The lead screw serves as the second transmission mechanism 430, which is connected to the blocking mechanism 450. Through rotation of the lead screw, a degree of opening/closing of the blocking mechanism 450 is adjusted, thus adjusting the beam spot size.

The central line of the blocking mechanism refers to a symmetry axis of the adjustment component. For example, the central line may be the symmetry axis of the particle beam.

The shielding plates may be flat panels, which may be in an open state (i.e., Off) or a closed state (i.e., On), used to not scatter or scatter the particle beam. The shielding plates are fixed to the lead screw, and their states are adjusted via the motion of the lead screw.

In some embodiments, the shielding plate may be one of the following shapes: L-shape, a square, a circle, a trapezoid, a triangle, a ring, a sector, or an irregular shape. A material of the shielding plate may include boron carbide, polycarbonate, or the like. A thickness of the shielding plate may be determined empirically. For example, the thickness of the shielding plate may be 0.01 mm, 0.02 mm, 0.03 mm, 0.05 mm, 0.1 mm, 0.2 mm, 0.3 mm, 0.5 mm, 1 mm, 2 mm, 3 mm, 5 mm, 10 mm, etc.

In some embodiments, as shown in FIG. 4, the beam spot size adjustment device is located downstream of a beam exit of the particle accelerator and includes the motor, a coupling 420, the lead screw, a bearing 440, and two independently controllable shielding plates. The coupling 420 refers to a device connecting two shafts or a device connecting a shaft with a rotating component. The bearing 440 may be a housed bearing.

In some embodiments, the motor drives the shielding plates to perform linear motion via the lead screw. When the shielding plate is in the open state, the particle beam can strike the shielding plates, causing scattering, so as to alter the beam spot size and beam energy. The beam spot size adjustment device may be used to increase the beam size to meet specific irradiation requirements. For example, two shielding plates (denoted as Shielding Plate 1 and Shielding Plate 2) can provide four beam spot size options: (1) Shielding Plate 1: OFF, Shielding Plate 2: OFF; (2) Shielding Plate 1: ON, Shielding Plate 2: OFF; (3) Shielding Plate 1: OFF, Shielding Plate 2: ON; (4) Shielding Plate 1: ON, Shielding Plate 2: ON. “OFF” indicates that the shielding plate is in the open state and does not scatter the beam, while “ON” indicates that the shielding plate is in the closed state, causing scattering of the beam, thereby affecting the beam spot size and energy.

In some embodiments of the present disclosure, the motor and the lead screw are adopted as the second drive mechanism 410 and the second transmission mechanism 430 respectively, which enables precise adjustment of the beam spot size. The beam spot size adjustment device 115 can accurately adjust the position of the shielding plates according to the second control instruction, thereby achieving precise control on the beam spot size of the particle beam. Both the motor and the lead screw are stable and reliable motion control components with excellent performance and longevity, ensuring stable operation and long-term service of the beam spot size adjustment device. Using the motor and the lead screw as the second drive mechanism 410 and second transmission mechanism 430, it facilitates rapid and efficient adjustment of beam spot size, contributing to improving radiotherapy efficiency and reducing treatment duration. The shielding plates are used as the blocking mechanism 450, which allows flexible adjustment of the beam spot size of the particle beam, enabling personalized treatment plans.

In some embodiments, as shown in FIG. 3, the particle accelerator 110 further includes a radiofrequency (RF) device 116, and/or a vacuum device 117, and/or a liquid cooling device 118. The RF device 116 may be configured to provide an accelerating electric field to the cavity 112. The vacuum device 117 may be configured to provide a vacuum environment for the cavity 112. The liquid cooling device 118 may be configured to cool the cavity 112 via a cooling liquid.

The RF device 116 is configured to provide the accelerating electric field to the cavity 112. The accelerating electric field is an electric field in the particle accelerator 110, which enables particles to gain kinetic energy and accelerate to a desired velocity for treatment. An RF electromagnetic field generated by the RF device 116 establishes the accelerating electric field.

In some embodiments, the RF device 116 may include one of a klystron, a superconducting RF cavity, or the like. The RF device 116 may generate the RF electromagnetic field, thereby establishing the accelerating electric field in the cavity 112.

The vacuum device 117 is configured to provide the vacuum environment for the cavity.

The liquid cooling device 118 is configured to cool the cavity 112 via a cooling liquid. In some embodiments, the liquid cooling device 118 may include one of a water-cooling plate, a water-cooling channel, a spray cooling system/a liquid helium dewar, or the like. The cooling liquid may include water, liquid nitrogen, or other cooling fluid.

In some embodiments of the present disclosure, the RF device enables particles to rapidly gain energy, thereby improving particle acceleration efficiency and reducing treatment time. The vacuum device maintains the cavity in a vacuum state, preventing collisions between gas molecules and the particles, and ensuring the stability and reliability of the transportation of the particle beam. The liquid cooling device cools the cavity to prevent the particle accelerator from damage due to overheating, enhancing the stability and reliability of the particle accelerator. These auxiliary devices (including the RF device, the vacuum device, and the liquid cooling device) ensure the stable operation and precise control of the particle accelerator, thereby improving the precision and accuracy of radiation therapy. Physicians can more precisely control the energy and the direction of the particle beam, enabling more personalized treatment plans and enhancing therapeutic outcomes.

Some embodiments of the present disclosure provide a method for operating an arc therapy apparatus (hereinafter referred to as the method), which may be used in any of the arc therapy apparatuses described in the present disclosure.

The arc therapy requires the arc therapy apparatus to have an arc rotation capability, i.e., the treatment gantry rotates around the subject (e.g., a patient), which allows the beam to rotate within a set angular range, achieving more precise irradiation shapes and dose distributions. Secondly, the arc therapy requires the arc therapy apparatus to have high-precision gantry positioning and rotation capabilities, including a precise gantry positioning system, a stable gantry rotation mechanism, and an accurate gantry position detection system, to ensure the accuracy and stability of the treatment gantry. Additionally, the arc therapy requires the arc therapy apparatus to be able to rapidly switch the energy of the particle beam during treatment. The treatment plan typically includes multiple arcs or angles, each of which may require different particle beam energy to meet specific therapeutic needs of the subject. The ability to rapidly switch energy enables the arc therapy apparatus to rapidly switch energy at each angle to achieve highly accurate dose distributions and treatment outcomes. Furthermore, the arc therapy requires that the arc therapy apparatus is equipped with an advanced treatment planning system and optimization algorithms, which is capable of generating a high-quality treatment plan based on an anatomical structure of the subject and dosage requirements.

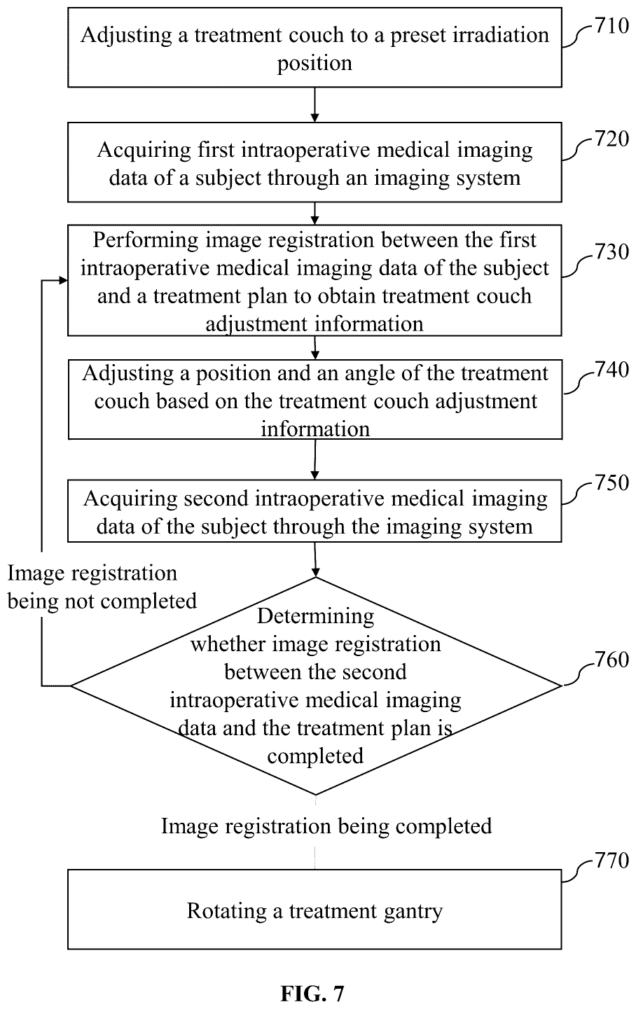

FIG. 6 is a flowchart of an exemplary process of a method for operating an arc therapy apparatus according to some embodiments of the present disclosure. In some embodiments, operations 610 and 620 may be performed by an operating device, as shown in FIG. 6. More descriptions regarding the operating device may be found in the related descriptions of FIG. 7.

In 610, a treatment plan may be acquired.

The treatment plan is a plan used for arc therapy.

In some embodiments, the treatment plan includes a plurality of preset angular intervals, and a preset dose of a particle beam corresponding to each of the preset angular intervals. The preset angular intervals are angular positions at which the particle beam irradiates a subject during the arc therapy.

In some embodiments, a physician and/or a treatment planning system (TPS) may determine the value and count of the preset angular intervals based on a location of a target region, morphology of the target region, a count of the target region, or the like. Embodiments of the present disclosure do not limit the count of the preset angular intervals. When the count of the preset angular intervals is large, the treatment gantry of the arc therapy apparatus can achieve a near-continuous rotation effect.

The preset dose of the particle beam corresponding to a preset angular interval refers to energy released by the particle beam irradiating at the preset angular interval. In some embodiments, the physician and/or the TPS may determine the preset dose of the particle beam corresponding to each of the preset angular intervals based on the location of the target region, a depth of the target region, the morphology of the target region, or the like.

In some embodiments, the treatment plan may be acquired in a variety of ways based on an actual condition of the subject. For example, the treatment plan may be developed by the physician and uploaded into a device (e.g., a control software system). As another example, the treatment plan may be determined by the TPS. More descriptions regarding the TPS may be found the related descriptions of FIGS. 1 and 2.

In the operation, the treatment plan may be loaded into the control software system of the arc therapy apparatus and parsed into machine setup information for corresponding configuration.

In 620, the treatment gantry of the arc therapy apparatus may be rotated to each of the preset angular intervals, respectively, to complete an irradiation process of the particle beam corresponding to each of the preset angular intervals.

In some embodiments, the operating device may control, based on the treatment plan, the treatment gantry to drive the particle accelerator and the beam delivery system to rotate to a first preset angular interval, and control the particle accelerator and the beam delivery system to release the corresponding preset dose of particle beam to the target region. After the irradiation of the particle beam corresponding to the first preset angular interval is completed, the operating device controls the treatment gantry to rotate to a second preset angular interval and complete the irradiation of the particle beam corresponding to the second preset angular interval. The above steps are repeated until the irradiation of the particle beam corresponding to all of the preset angular intervals is completed, and the arc therapy is completed.

In some embodiments of the present disclosure, by acquiring the treatment plan, the arc therapy apparatus can accurately set the required treatment dose at each of the preset angular intervals. The rotating treatment gantry allows for multi-angle irradiation, covering a wide treatment range to maximize irradiation of the tumor while reducing the radiation dose to normal tissues to maximize the protection of the subject's health. In addition, compared to fixed-angled radiation therapy, the arc therapy can better accommodate variations in tumor morphology and difference of the anatomical structure of the subject, enhancing treatment accuracy and efficacy for improved tumor control. In summary, the method is efficient, precise, and comprehensive. Its implementation in the arc therapy apparatus contributes to superior clinical outcomes for the subject while elevating overall treatment quality and safety.

In the arc therapy apparatus, before performing the arc therapy (e.g., before rotating the treatment gantry), it is first necessary to determine the irradiation position of the subject, which is achieved by adjusting the treatment couch to a preset irradiation position. The treatment couch may be translated, rotated, and raised/lowered in multiple axes for achieving precise position adjustments to ensure that the target region of the subject is located at the correct irradiation position.

FIG. 7 is a flowchart of an exemplary process for adjusting a treatment couch according to some embodiments of the present disclosure.

In some embodiments, as shown in FIG. 7, the operating device further performs the following operations 710 to 740 before rotating the treatment gantry (i.e., before performing operation 620).

In 710, the treatment couch may be adjusted to a preset irradiation position.

The preset irradiation position refers to a pre-defined irradiation position of the subject in the treatment plan. The preset irradiation position is the position of the subject on the treatment couch to ensure that the particle beam accurately irradiates the target region.

In some embodiments, the preset irradiation position may be set based on experience. In some embodiments, the operating device may translate, rotate, and raise/lower the treatment couch in a plurality of axes to ensure that the treatment couch is located at the preset irradiation position.

In 720, first intraoperative medical imaging data of the subject may be acquired through an imaging system.

The intraoperative medical imaging data refers to medical imaging data of the subject acquired during the arc therapy. For example, the intraoperative medical imaging data may include one of an X-ray image, a Computed Tomography (CT) scan image, an Magnetic Resonance (MR) scan image, or the like. The intraoperative medical imaging data may be acquired in real time by the imaging system.

The first intraoperative medical imaging data refers to the intraoperative medical imaging data of the subject acquired before the particle beam irradiates the target region.

In 730, image registration may be performed between the first intraoperative medical imaging data of the subject and the treatment plan to obtain treatment couch adjustment information.

The image registration refers to a process of aligning and matching the first intraoperative medical imaging data with the treatment plan. In some embodiments, the image registration may involve comparing the target region in the first intraoperative medical imaging data with a plurality of preset angular intervals in the treatment plan to determine whether the target region is within the plurality of preset angular intervals.

In response to determining that the target region is within the plurality of preset angular intervals, the image registration is completed, and the operating device performs operation 620. In response to determining that the target region is not within at least one of the plurality of preset angular intervals, the operating device generates the treatment couch adjustment information.

The treatment couch adjustment information refers to information related to adjustment of the treatment couch. In some embodiments, the treatment couch adjustment information may include a direction, an angle, a distance, etc., that the treatment couch needs to be adjusted to.

In some embodiments, the operating device may determine the treatment couch adjustment information based on a result of the image registration. For example, if the target region is not within at least one of the plurality of preset angular intervals, the operating device may determine the direction, the angle, and the distance that the treatment couch needs to be adjusted to when the target region is located in each of the plurality of preset angular intervals, and generate the treatment couch adjustment information.

In 740, a position and an angle of the treatment couch may be adjusted based on the treatment couch adjustment information.

In some embodiments, the operating device may determine the direction, the angle, and the distance that the treatment couch needs to be adjusted to based on the treatment couch adjustment information, and translate, rotate, and raise/lower the treatment couch in multiple axes.

In some embodiments of the present disclosure, adjusting the treatment couch to the preset irradiation position can ensure that clear first intraoperative medical imaging data is acquired, facilitating the subsequent image registration between the first intraoperative medical imaging data and the treatment plan, ensuring precision and accuracy of the subsequent treatment process. The first intraoperative medical imaging data acquired by the imaging system can provide the physician with detailed information about the anatomical structure of the subject, helping the physician to determine the location, the size, and the shape of the tumor, and the structure of surrounding tissues for determining an accurate treatment plan. The image registration aligns the intraoperative medical imaging data with the treatment plan, ensuring accurate implementation of the treatment plan and providing precise positioning information. As a result, the precision and accuracy of the arc therapy are ensured, maximizing radiation delivery to the tumor while minimizing damage to normal tissues.

In the arc therapy, after adjusting the position and the angle of the treatment couch, it is necessary to reacquire intraoperative medical imaging data (i.e., second intraoperative medical imaging data) of the subject in order to ensure the accuracy of the arc therapy. This is because minor changes may occur in the anatomical structure of the subject after the treatment couch is re-positioned and re-angled, and these changes may affect the accuracy of the treatment plan. Therefore, it is necessary to re-acquire the intraoperative medical imaging data (i.e., the second intraoperative medical imaging data) of the subject, and determine whether the image registration is completed. If the image registration is completed, the treatment gantry may be rotated to the first preset angular interval. If the image registration is not completed, the treatment couch adjustment information may be re-acquired to re-adjust the treatment couch until the image registration is completed accurately, thereby ensuring irradiation accuracy of the particle beam and the effectiveness of the arc therapy.

In some embodiments, as shown in FIG. 7, after adjusting the position and the angle of the treatment couch, the following operations 750-770 may be performed. It may be understood that operations 750-770 are not necessary, i.e., if the operating device has correctly adjusted the position and the angle of the treatment couch after executing operations 710-740, the process may be directly ended, and if the operating device has not correctly adjusted the position and the angle of the treatment couch after executing operations 710-740, the operating device executes operations 750-770 to readjust the position and the angle of the treatment couch.

In 750, the second intraoperative medical imaging data of the subject may be acquired through the imaging system.

The second intraoperative medical imaging data refers to the intraoperative medical imaging data of the subject that is acquired after adjusting the position and the angle of the treatment couch.

In 760, it may be determined whether image registration between the second intraoperative medical imaging data and the treatment plan is completed.

More descriptions regarding the image registration may be found in operation 730 and the related descriptions thereof.

In some embodiments, in response to determining that the image registration is completed, operation 770 is performed; in response to determining that the image registration is not completed, the treatment couch adjustment information is re-acquired to re-adjust the treatment couch (i.e., operations 730 to 760 are re-executed) until the image registration is completed.

If the image registration is completed, i.e., the intraoperative medical imaging data is accurately aligned to the treatment plan, then the treatment gantry is rotated to the first preset angular interval. Rotating the treatment gantry is a key step in the implementation of the arc therapy. By rotating the treatment gantry, the particle accelerator and the beam delivery system rotate around the isocenter to achieve the arc therapy.

If the image registration is not completed, i.e., the intraoperative medical imaging data is not accurately aligned to the treatment plan, the position and the angle of the treatment couch are required to be further adjusted. Therefore, the treatment couch adjustment information may be re-acquired to re-adjust the position and the angle of the treatment couch to ensure that the irradiation position and posture of the subject are precisely in place for accurate irradiation of the particle beam.

The above adjustment process may be iteratively repeated until the image registration between the intraoperative medical imaging data and the treatment plan is completed.

In 770, the treatment gantry may be rotated.

In some embodiments of the present disclosure, after each adjustment of the position and the angle of the treatment couch, the intraoperative medical imaging data is re-acquired and image registration is performed between the intraoperative medical imaging data and the treatment plan. The arc therapy is performed after the image registration is completed, which prevents minor displacements or deformations of the target region of the patient from affecting treatment precision and safety, thereby further improving therapeutic efficacy. By determining whether the image registration between the intraoperative medical imaging data and the treatment plan is completed, it can provide timely feedback, ensuring the real-time and accuracy of the treatment plan to guide subsequent treatment steps. If the image registration is not completed, the treatment couch adjustment information is re-acquired to make further adjustments to ensure that the irradiation position and angle of the subject are precisely in place, ultimately enhancing the accuracy and effectiveness of the arc therapy. In summary, the real-time and precision of the method ensure the safety and efficacy of the arc therapy, providing the subject with high-level personalized treatment that minimizes complication risks while improving overall quality and success rate of the treatment.

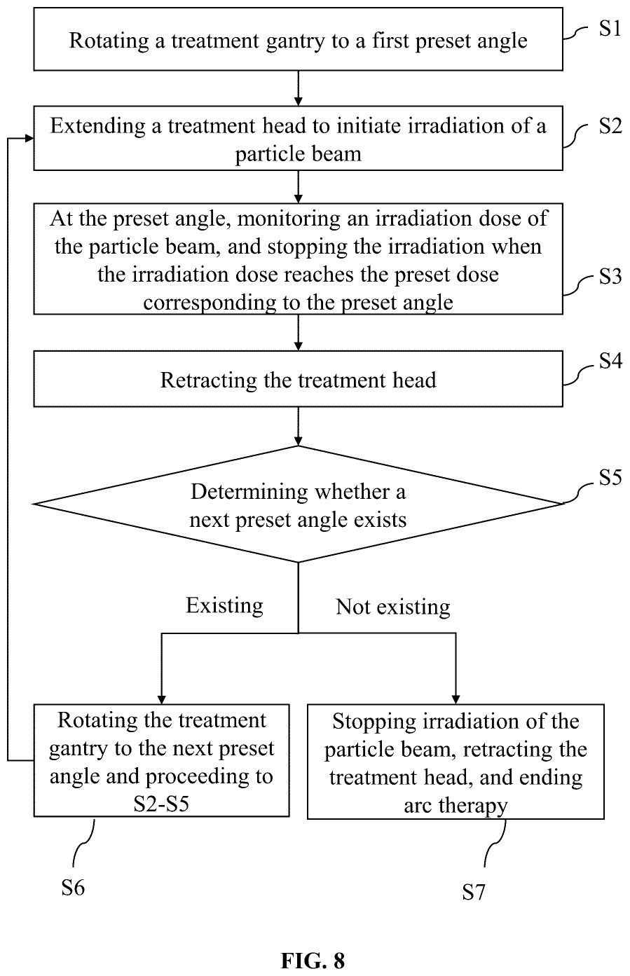

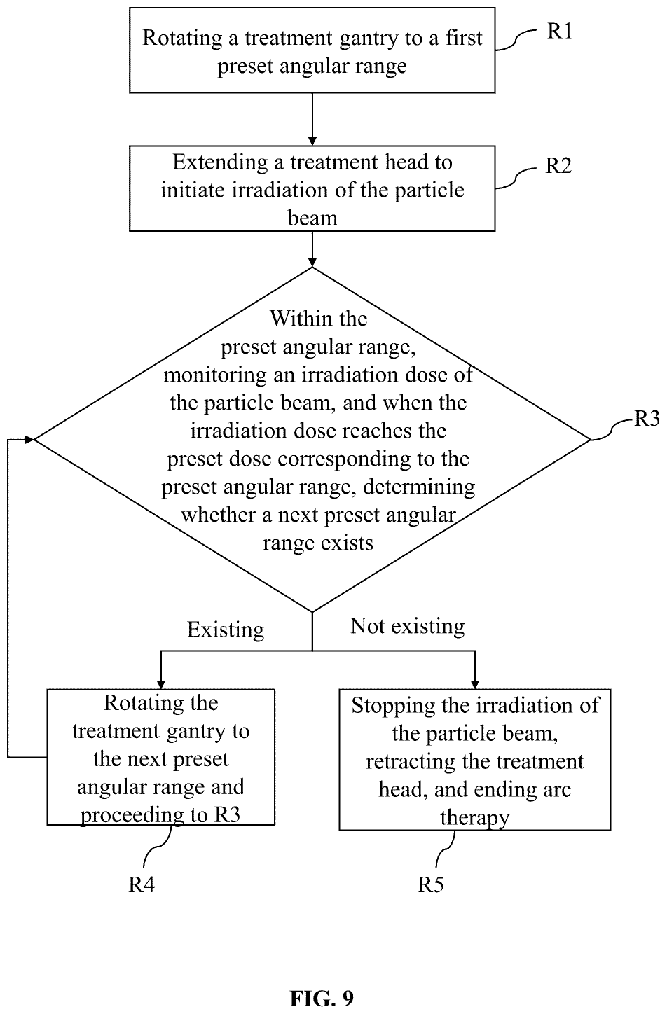

In some embodiments, the arc therapy may adopt a first implementation manner in which the preset angular intervals are point values or a second implementation manner in which the preset angular intervals are range values. The two implementation manners are described below in connection with FIGS. 8 and 9.

FIG. 8 is a flowchart of an exemplary process of a first implementation manner for achieving arc therapy according to some embodiments of the present disclosure.

In some embodiments, the preset angular intervals are expressed as point values, denoted by discrete preset angles, and the operating device performs the following operations during the arc therapy (i.e., operations 620 may include sub-operations S1-S7).

S1, the treatment gantry may be rotated to a first preset angle.

S2, a treatment head may be extended to initiate irradiation of the particle beam.

S3, at the preset angle, an irradiation dose of the particle beam may be monitored, and the irradiation may be stopped when the irradiation dose reaches the preset dose corresponding to the preset angle.

During the irradiation of the particle beam at each preset angle, based on the preset dose, the irradiation dose of the particle beam at the current preset angle is monitored (i.e., the irradiation dose corresponding to each preset angle is counted independently). After the particle beam delivers a required dose, the irradiation is stopped automatically to ensure that the irradiation dose is accurate. This mechanism guarantees that the irradiation dose of the particle beam complies with requirements of the treatment plan and prevents overexposure or over irradiation. In this embodiment, the irradiation dose of the particle beam reaching the preset dose corresponding to the preset angle means that the irradiation dose is not less than the preset dose. In other words, when the detected irradiation dose of the particle beam is equal to the preset dose, or an absolute difference between the detected irradiation dose of the particle beam and the preset dose is not greater than a preset value, the particle accelerator immediately stops emitting the particle beam. The embodiments of the present disclosure impose no limitations on the detection manners of the irradiation dose. Merely by way of example, the irradiation dose may be detected through an ionization chamber in the treatment head or through a detection device based on Cherenkov radiation.

S4, the treatment head may be retracted.

S5, it may be determined whether a next preset angle exists. In response to determining that the next preset angle exists, proceeding to S6; in response to determining that the next preset angle does not exist, proceeding to S7.

If the next preset angle exists, the next operation (i.e., S6) is executed. If not, it indicates that the irradiation of all preset angles is completed, and the arc therapy is completed.

In some embodiments, the operating device may detect whether the next preset angle exists by comparing a previous preset angle and the treatment plan. If the next preset angle exists, proceeding to S6; if not, proceeding to S7.

S6, the treatment gantry may be rotated to the next preset angle, and proceeding to S2-S5.

In S6, the treatment gantry may be rotated to the next preset angle, the treatment head may be extended again, and the irradiation of the particle beam at the next preset angle may be initiated. In this way, the arc therapy apparatus gradually complete the irradiation at all the preset angles, realizing multi-angle arc therapy.

During the irradiation at each preset angle, the continuity of the arc therapy is ensured through the sequential operations of extending the treatment head, delivering the particle beam irradiation, stopping the beam delivery, retracting the treatment head, and detecting whether the irradiation is complete. That is to say, the treatment head undergoes extension and retraction for multiple times throughout the arc therapy.

S7, the irradiation of the particle beam may be stopped, the treatment head may be retracted, and the arc therapy may be ended.

When the irradiation at all of the preset angles are completed, the arc therapy is completed.

In some embodiments of the present disclosure, with the preset angles and the preset doses, the arc therapy apparatus is able to automatically complete the rotation of the treatment gantry and the irradiation of the particle beam without manual intervention, which improves the efficiency and consistency of the arc therapy. In addition, when the preset angular intervals are represented as point values (e.g., the discrete preset angles), by retracting/extending the treatment head before and after the rotation of the treatment gantry, it ensures that the particle beam can be accurately irradiated to the target region (e.g., the lesion region) of the subject at each preset angle, avoiding irradiation deviation and error to ensure the accuracy and efficacy of the arc therapy. After the irradiation at each preset angle is completed, the treatment head is retracted to avoid the risk of collision between the treatment head and the subject or collision between the treatment head and device, and the retraction and expansion control of the treatment head and the application of the safety interlocking system safeguard the safety and stability of the arc therapy. Through the setting of the irradiation dose, it ensures that the particle accelerator stops delivering the particle beam after the irradiation dose reaches the required dose, thus ensuring the precise control of the irradiation dose of the particle beam, avoiding overexposure, reducing harm to the subject, and safeguarding the safety and effectiveness of the arc therapy. Independent irradiation of the particle beam at each preset angle ensures that irradiation parameters can be flexibly adjusted according to the actual conditions and treatment requirements of the subject to achieve personalized treatment. Moreover, the method allows irradiation at different preset angles to realize all-around irradiation at multiple angles, improving the precision and therapeutic efficacy of the arc therapy.

FIG. 9 is a flowchart of an exemplary process of a second implementation manner for achieving arc therapy according to some embodiments of the present disclosure.

In some embodiments, the preset angular intervals are represented by range values, e.g., angular ranges. The operating device may perform the following operations during the arc therapy (i.e., operation 620 may include at least one of sub-operations R1-R5).

R1, the treatment gantry may be rotated to a first preset angular range.

R2, the treatment head may be extended to initiate irradiation of the particle beam.

In some embodiments, an extension position of the treatment head does not exceed a preset position, which is determined based on a preset collision constraint condition.

The preset position is a position where no collision occurs. For example, the preset position may be a position where the treatment head, when extended, does not collide with the subject and/or the treatment couch.

The collision constraint condition refers to a constraint condition that prevents collisions of the treatment head. In some embodiments, the collision constraint condition may include that a distance between the treatment head and the subject is not less than a first distance, and/or a distance between the treatment head and the treatment couch is not less than a second distance. The first distance and the second distance may be the same or different, and the specific values thereof may be set by a person skilled in the art based on experience.

During the arc therapy, the arc therapy apparatus automatically controls the extension position of the treatment head to ensure that the extension position of the treatment head does not exceed the preset position, which maintains the appropriate distance between the treatment head and the target region and ensures that the particle beam is correctly irradiated to the target region of the subject, thereby enhancing treatment precision and reducing damages of side effects to normal tissues. By preventing collisions between the treatment head and the subject, unexpected damages and malfunctions of the arc therapy apparatus can be minimized, thereby reducing maintenance and repair costs and enhancing treatment safety and stability. The preset collision constraint condition ensures that the extension position of the treatment head always remains within the safety range, decreasing the need for adjustments and treatment interruptions (e.g., pauses required to readjust or repair the treatment head due to collisions), thus improving treatment efficiency and workflow continuity. The automated control of the extension position of the treatment head eliminates manual intervention, simplifies operational procedures, reduces operator workload and potential errors, and improves overall usability of the arc therapy apparatus.

R3, within the preset angular range, an irradiation dose of the particle beam may be monitored, and when the irradiation dose reaches the preset dose corresponding to the preset angular range, it may be determined whether a next preset angular range exists. In response to determining that the next preset angular range exists, proceeding to R4; in response to determining that the next preset angular range does not exist, proceeding to R5.

R4, the treatment gantry may be rotated to the next preset angular range and proceeding to R3.

R5, the irradiation of the particle beam may be stopped, the treatment head may be retracted, and the arc therapy may be ended.

In some embodiments of the present disclosure, when the preset angular intervals are represented by the angular range during the arc therapy, the treatment head does not need to be retracted after irradiation in each angular range, i.e., the treatment head avoids repeated extension/retraction movements, which saves operational time during treatment, thereby reducing total treatment duration and improving treatment efficiency. Additionally, there is no need to adjust the position of the treatment head before or after rotating the treatment gantry, which simplifies the workflow, reduces operational steps, lowers operational complexity, and enhances treatment convenience and efficiency. This approach also improves the stability and reliability of the arc therapy apparatus. Furthermore, under this implementation manner, the maximum extension position of the treatment head throughout the treatment plan can be pre-determined and used as a limit to constrain its movement, which prevents collisions between the treatment head and the subject or collisions between the treatment head and device while avoiding unnecessary adjustments during rotation, ultimately increasing treatment efficiency and shortening the overall treatment duration.

As an example, in the first implementation manner, preset angles 0°, 45°, 90°, 135° and 180° correspond to preset doses of 10 Monitor Units (MU), 10 MU, 10 MU, 10 MU, and 10 MU, respectively. In the second implementation manner, preset angular ranges 5°-22.5°, 45°+22.5°, 90°+22.5°, 135°+22.5°, and 157.5°-185° correspond to preset doses of 10 MU, 10 MU, 10 MU, 10 MU, and 10 MU, respectively. In the second implementation manner, the particle beam maintains continuous irradiation during both the detection operation and the treatment gantry rotation operation, ensuring the total delivered dose remains consistent with that of the first implementation manner.

In the first implementation manner, the preset dose at each of the preset angles is 10 MU, indicating that at each of the preset angles, after the treatment gantry is rotated to the preset angle, the treatment head extends to initiate irradiating the particle beam until the irradiation dose of the particle beam reaches 10 MU, then the irradiation is stopped and the treatment head is retracted. Irradiation is performed independently at each preset angle with the same irradiation dose of 10 MU.

In the second implementation manner, the preset doses correspond to the preset angular ranges are set as 10 MU, 10 MU, 10 MU, 10 MU, and 10 MU. In the second implementation manner, the treatment gantry continuously irradiates during rotation, thus, the irradiation between adjacent preset angular ranges is continuous without requiring separate treatment head retractions for each range.

In the first implementation manner, each preset angle interval represents an independent irradiation process where the treatment head retracts after each angle, ensuring both the accuracy and safety of irradiation. The first implementation manner is particularly suitable for treatment scenarios requiring high irradiation precision, especially for subjects with complex shapes of the target region and needing fine adjustments to dose distribution, enabling more personalized treatment. In the second implementation manner, continuous irradiation reduces the number of extension and retraction of the treatment head during the treatment process, simplifying operational procedures and improving treatment efficiency, thereby helping to reduce the total treatment duration. While minimizing the treatment head's extension/retraction steps, the second implementation manner also reduces the risk of collision between the treatment head and the subject or collision between the treatment head and other devices, enhancing treatment safety, making it suitable for general radiotherapy, particularly for large-area and regularly shaped target regions, enabling more efficient treatment.

In summary, both the first implementation manner (e.g., sub-operations S1-S7) and the second implementation manner (e.g., sub-operations R1-R5) have their respective advantages and applicable scenarios. The choice of implementation manner depends on the performance of the treatment machine and the actual conditions of the subject. By selecting the appropriate implementation manner based on the actual conditions and treatment requirements of the subject, it can enable more personalized and efficient arc therapy. For scenarios requiring more precise adjustments and personalized treatment, the first implementation manner (e.g., sub-operations S1-S7) may be more suitable; while for scenarios prioritizing high efficiency and simplified workflows, the second implementation manner (e.g., sub-operations R1-R5) may be preferable. In clinical practice, physicians may flexibly select the appropriate implementation manner according to the subject's condition and treatment objectives to achieve optimal therapeutic outcomes. Furthermore, for the second implementation manner, by pre-determining the preset dose for each angular interval, it ensures consistent total irradiation dose, guaranteeing treatment accuracy and therapeutic efficacy.

In some embodiments, if a count of the preset angular intervals is large (for example, more than 30, such as 90, 180, 360, or 720), the second implementation manner can achieve a stepless control process approximating continuous rotation and continuous irradiation, thereby producing a qualitative leap through quantitative changes. The beneficial effects include as follows.

1. Smooth and continuous irradiation: as the count of preset angular intervals increases, the irradiation becomes smoother and more continuous, with nearly seamless movement of the particle beam and no apparent gaps, which enables more uniform dose distribution, thereby avoiding discontinuities in dose distribution and improving irradiation accuracy and homogeneity.

2. High treatment efficiency: with 360 or more preset angular intervals, there is no need to retract the treatment head after irradiation at each interval, significantly reducing the number of extension/retraction movements during treatment and simplifying operational procedures. Simultaneously, as the treatment gantry rotates continuously without noticeable pauses, treatment efficiency is substantially improved, helping to shorten overall treatment duration and alleviating discomfort and anxiety for the subject.

3. Precise dose control: the increased count of preset angular intervals allows the continuous irradiation at different angles to better compensate for dose distribution inhomogeneity. Through pre-determination of preset doses, a consistent total irradiation dose is ensured, enabling precise dose control to avoid overexposure or underexposure and guaranteeing treatment safety and effectiveness.

4. Personalized treatment planning: when the count of preset angular intervals is large, treatment planning flexibility is enhanced. Physicians can adjust the count and spacing of preset angular intervals according to the specific conditions and treatment requirements of the subject to achieve more personalized treatment. Different tumor types and locations may require different counts of preset angular intervals, and such adjustments can better accommodate diverse treatment requirements.

5. Improved robustness: the implementation manner with a large count of preset angular intervals can enhance the robustness of the treatment plan. Even if uncertainties in image registration or other technical procedures exist at an angle, the continuous gantry rotation and irradiation characteristics ensure that the overall treatment process remains stable and accurate, without significant dose errors caused by any angle.

Some embodiments of the present disclosure further present an operating device. The operating device is configured to operate and execute the arc therapy apparatus and perform the method for operating the arc therapy apparatus described in any embodiment of the present disclosure.

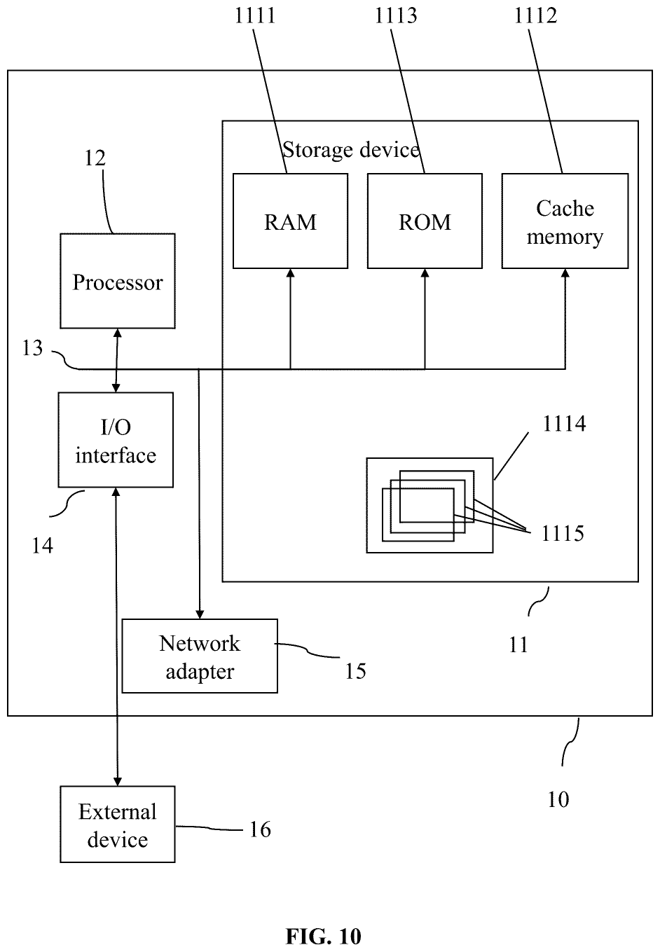

FIG. 10 is a structural block diagram of an operating device according to some embodiments of the present disclosure. In some embodiments, as shown in FIG. 10, an operating device 10 may include one or more storage devices 11, one or more processors 12, a bus 13 connecting different platform systems, one or more input/output (I/O) interfaces 14, and one or more network adapters 15.

The storage device 11 is configured to store various data and instructions for operation of the operating device. In some embodiments, the storage device 11 may include a computer-readable medium in the form of volatile memory. For example, the storage device 11 may include at least one of random access memory (RAM) 1111, cache memory 1112, read-only memory (ROM) 1113, or the like.

In some embodiments, the storage device 11 may include a utility 1114 having one or more program modules 1115. Each of the one or more program modules 1115 includes an operating system, one or more applications, other program modules, program data, or any combination thereof.

In some embodiments, the storage device 11 also stores a computer program, which may be read by the processor 12 to cause the processor 12 to implement any operation of the method for operating the arc therapy apparatus described in any embodiment of the present disclosure.

The processor 12 is configured to read various data and instructions and perform related operations. For example, the processor 12 may read and execute the aforementioned computer program and the utility 1114.

In some embodiments, the processor 12 may include at least one of an application-specific integrated circuit (ASIC), a digital signal processor (DSP), a programmable logic controller (PLC), a complex programmable logic device (CPLD), a field-programmable gate array (FPGA), or the like.

The bus 13 is configured to implement communication connections and data interactions for various structures in the operating device. In some embodiments, the bus 13 may include one of a memory bus, a memory controller, a peripheral bus, a graphics acceleration port, or the like, or any combination thereof.

In some embodiments, the one or more I/O interfaces 14 are communicatively connected with one or more external devices 16. In some embodiments, the operating device 10 is communicatively connected to one or more external devices 16 (e.g., a keyboard, a pointing device, a Bluetooth device, a router, a modem, etc.) based on the one or more I/O interfaces 14 for data interaction.

The network adapter 15 is configured to enable the operating device 10 to connect to a network. In some embodiments, the operating device 10 may be connected to one or more networks (e.g., a local area network (LAN), a wide area network (WAN), the Internet, etc.) via the network adapter 15. The network adapter 15 may communicate with other modules of the operating device 10 via the bus 13.

Furthermore, it may be understood that, although not shown in the drawings, in practical applications, the operating device 10 may incorporate other hardware and/or software modules such as a microcode, a device driver, a redundant processor, an external disk drive array, a redundant array of independent disk (RAID), a tape drive, a data backup storage platform, or the like.

Some embodiments of the present disclosure further provide a magnetic field adjustment device.

In some embodiments, the magnetic field adjustment device is applied to a particle accelerator mounted on a treatment gantry of an arc therapy apparatus. The particle accelerator rotates along with the treatment gantry during rotation of the treatment gantry. The particle accelerator includes a magnetic field providing device configured to provide a particle deflection magnetic field. The magnetic field adjustment device is configured to adjust the particle deflection magnetic field based on a change of a gantry angle of the treatment gantry, such that a particle beam generated by a particle radiation source satisfies a preset particle beam condition under the particle deflection magnetic field. The preset particle beam condition indicates a preset range of each of one or more of an energy level, a beam spot size, and a beam spot position of the particle beam.

In some embodiments, the particle accelerator is applied to the arc therapy apparatus and rotates along with the treatment gantry.

The gantry angle refers to an angle of the treatment gantry. By way of example, the treatment gantry includes two arms and a gantry body between the arms. The two arms are pivotally coupled to a fixed base respectively, and the particle accelerator is disposed on the gantry body. For example, a zero-degree position may be defined as a longitudinal direction (or a length direction) of the arms being parallel to a treatment couch, and the gantry angle refers to a rotational angle of the arms around their respective rotation axis.

The particle beam condition refers to ranges of parameters such as the energy level, the beam spot size, and the beam spot position of the particle beam. The particle beam condition is used to preset and guide the radiation therapy.

The beam spot size and the beam spot position determine a correct irradiation position and a correct irradiation shape of the particle beam.

The beam spot position refers to a center of a dose distribution of the particle beam on a plane perpendicular to a beam reference axis.

In some embodiments, the beam spot position requires to be precisely controlled to ensure that the beam spot position remains targeted at a center of a tumor region, maintaining this accuracy even during gantry rotation, thereby guaranteeing uniform dose delivery across the entire tumor region.

The present disclosure does not limit the preset ranges of the parameters such as the energy level, the beam spot size, the beam spot position, etc., of the particle beam, which may be determined based on actual conditions of a subject, treatment requirements, and a treatment plan. Further descriptions may be found in the previous related descriptions.

The magnetic field adjustment device provided by the embodiments of the present disclosure enables the particle deflection magnetic field to be adjusted based on the change of the gantry angle of the treatment gantry, effectively compensating for magnetic field changes caused by factors such as gravity. Through the adjustments made by the magnetic field adjustment device, the particle deflection magnetic field in the particle accelerator can remain stable during the rotation of the treatment gantry, ensuring that the particle beam generated by the particle radiation source meets the particle beam condition and maintains parameters such as the energy level, the beam spot size, and the beam spot position of the particle beam within acceptable ranges. The magnetic field adjustment device features an automatic adjustment function, which allows for rapid magnetic field adjustments based on the gantry angle, thereby making the adjustment of the magnetic field more intelligent and efficient, reducing the workload of operators. Furthermore, the particle beam condition may be preset according to the actual conditions of the subject and the treatment plan, ensuring more personalized treatment. The automatic adjustment function enables the arc therapy to better adapt to the needs of different subjects (e.g., patients).

FIG. 11 is a structural block diagram of a magnetic field adjustment device according to some embodiments of the present disclosure.

In some embodiments, as shown in FIG. 11, the magnetic field adjustment device 114 includes a detection component 1110, a control component 1120, and at least one execution component 1130. The detection component 1110 is mounted inside a particle accelerator and is configured to detect magnetic field information of a particle deflection magnetic field. The control component 1120 is configured to determine motion control information of a magnetic field providing device 113 based on the magnetic field information and a preset magnetic field condition and generate a first control instruction corresponding to each of the at least one execution component 1130 based on the motion control information. The preset magnetic field condition is determined based on a preset particle beam condition. Each of the at least one execution component 1130 is connected to the control component 1120 and the magnetic field providing device 113 and is configured to adjust a position and/or an orientation of the magnetic field providing device 113 based on the first control instruction corresponding to the execution component.

The detection component 1110 refers to a component for detecting the magnetic field information of the particle deflection magnetic field.

The control component 1120 refers to a core processing unit in the magnetic field adjustment device 140. In some embodiments, the control component 1120 may include a high-performance processor, a signal processing module, and a communication interface, enabling efficient collaboration with the detection component and the execution component. The execution component 1130 is a mechanical component of the magnetic field adjustment device 140 that is responsible for physically adjusting the position and/or the orientation of the magnetic field providing device 113. Each of the at least one execution component 1130 is located in a portion or region of the particle accelerator, and a mounting base of each of the at least one execution component 1130 is relatively stationary in relation to the treatment gantry. For example, the mounting base of each of the at least one execution component 1130 is detachably installed on the treatment gantry.

In some embodiments, each of the at least one execution component 1130 is configured to adjust, based on the first control instruction corresponding to the execution component, the magnetic field providing device 113 that generates the particle deflection magnetic field. When two or more execution components 1130 are provided, the execution components 1130 may work together to adjust the position and/or the orientation of the magnetic field providing device 113 to cause the particle deflection magnetic field to satisfy the preset magnetic field condition.

The embodiments of the present disclosure do not limit the count of the at least one execution component 1130. For example, the count of the at least one execution component 1130 may be 1, 2, 3, 4, 6, 8, 12, or the like.

Merely by way of example, if the count of the at least one execution component 1130 is 2, and the two execution components may be provided on two sides of the magnetic field providing device, with one execution component on each side.

As another example, if the count of the at least one execution component 1130 is 4, two execution components may be provided on each side of the magnetic field providing device. The two execution components 1130 on the same side may be arranged such that longitudinal directions of the two execution components 1130 are parallel or non-coplanar (including the special case of skew orthogonal).

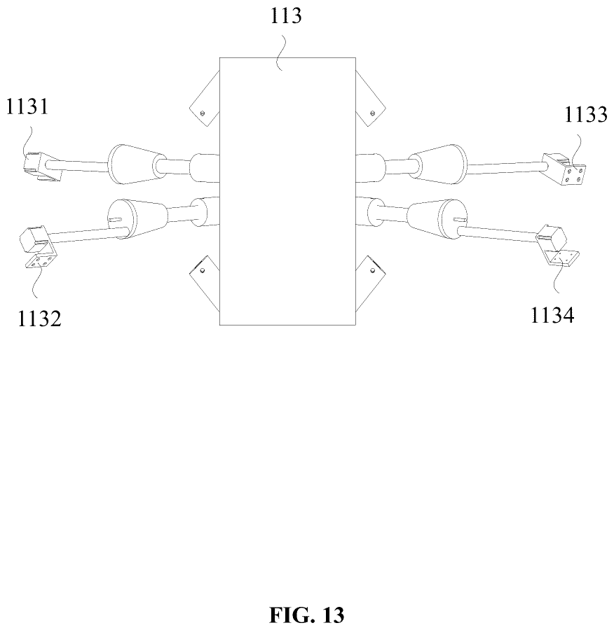

More descriptions regarding the execution component 1130 may be found in the related descriptions of FIGS. 13-16.

The magnetic field information refers to a collection of data describing a state of the particle deflection magnetic field. For example, the magnetic field information may include a field strength of the particle deflection magnetic field, a field direction of the particle deflection magnetic field, and other factors that may affect the path and features of the particle beam.

More descriptions regarding the particle deflection magnetic field may be found in the related descriptions of FIG. 8.

The preset magnetic field condition defines preset ranges of magnetic field parameters such as the field direction and the field strength of the particle deflection magnetic field. For example, the preset magnetic field condition may include the field strength, the field direction, or the like.

In some embodiments, the preset magnetic field condition may be determined based on the particle beam condition. In some embodiments, the preset magnetic field condition may be determined based on an actual clinical treatment plan, incorporating a physician's professional advice and actual conditions of the subject. More descriptions regarding the particle beam condition may be found in the related descriptions above.

The motion control information refers to a set of instructions for directing the at least one execution component 1130 to adjust the position and/or the orientation of the magnetic field providing device 113. For example, if a current field strength is detected to be lower than a preset value, the motion control information may instruct an execution component to move its connected magnetic field providing device upward by 1 cm and rotate the magnetic field providing device clockwise by 5 degrees to enhance the field strength.

In some embodiments, the control component 1120 may determine a difference between the magnetic field information provided by the detection component 1110 and the preset magnetic field condition and determine a required adjustment magnitude and a required adjustment direction using a built-in algorithm based on the difference, thereby generating the motion control information.

In some embodiments, the control component 1120 may determine an optimal adjustment strategy based on the difference between the magnetic field information provided by the detection component 1110 and the preset magnetic field condition. Based on the optimal adjustment strategy, the control component 1120 may generate the first control instruction corresponding to each of the at least one execution component 1130 in a customized manner, ensuring that all adjustments work in coordination to jointly achieve optimal performance of the particle beam.

In some embodiments, the at least one execution component 1130 receives the first control instruction(s) from the control component 1120, and drives the magnetic field providing device to perform fine adjustments such as translation, rotation, etc., to dynamically regulate the particle deflection magnetic field, ensuring that the particle beam meets the particle beam condition.

In the embodiment of the present disclosure, the magnetic field adjustment device can precisely adjust the particle deflection magnetic field by combining the real-time magnetic field monitoring acquired by the detection component with the motion control information determined by the control component. This approach ensures that the particle beam generated by the particle radiation source satisfies the particle beam condition, thereby improving the accuracy of the arc therapy. The control component determines the motion control information based on the magnetic field information and the preset magnetic field condition, enabling the first control instruction corresponding to each of the at least one execution component to dynamically adjust the particle deflection magnetic field in real time. This ensures that parameters such as the energy level, the beam spot size, and the beam spot position of the particle beam remain within acceptable ranges, thereby ensuring the stability and consistency of the arc therapy. The control component autonomously determines the motion control information based on the magnetic field information and generate the first control instruction(s) corresponding to the execution component(s), making the adjustment of the particle deflection magnetic field more intelligent and efficient, and reducing manual labor costs.

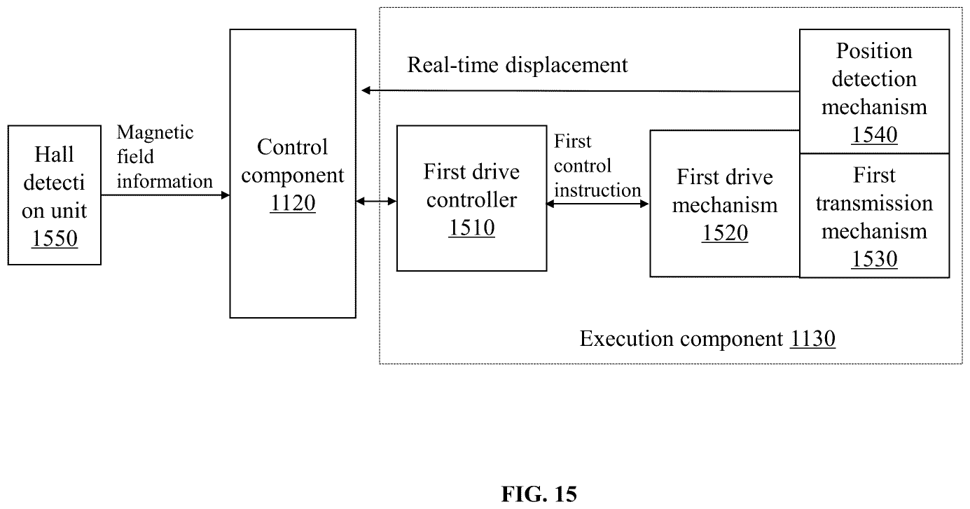

In some embodiments, as shown in FIG. 15, the detection component 1110 includes at least one Hall detection unit 1550, which is configured to detect a magnetic field at at least one location of the particle deflection magnetic field to obtain the magnetic field information.

The Hall detection unit 1550 refers to a sensor designed based on the Hall effect principle for accurately measuring a strength and a direction of a magnetic field. Each of the at least one Hall detection unit 1550 is configured to detect the magnetic field at one location of the particle deflection magnetic field in a particle accelerator to obtain the magnetic field information at the location through the Hall effect. For example, the Hall detection unit may be a Hall probe. The Hall detection unit is arranged inside the particle accelerator or at another location capable of detecting the particle deflection magnetic field, to sense a change in the magnetic field.

When the Hall detection unit 1550 is placed at a location in the particle accelerator, if a magnetic field is present, the Hall detection unit 1550 generates a voltage difference that is related to the strength of the magnetic field and the direction of the magnetic field, so that the magnetic field information (e.g., a magnetic flux density) at the location may be obtained.

The present disclosure does not limit the count of the at least one Hall detection unit 1550. The magnetic field adjustment device 140 may determine the strength and direction of the magnetic field at different locations, thereby obtaining the magnetic field information at the at least one location of the particle deflection magnetic field.

In some embodiments of the present disclosure, the at least one Hall detection unit enables accurate measurement of the magnetic field information of the particle deflection magnetic field, ensuring that the magnetic field adjustment device can acquire high-quality magnetic field data, which provides a reliable basis for subsequent adaptive control. When a plurality of Hall detection units are provided, magnetic field changes can be detected simultaneously at different locations. By performing detection at multiple locations, the magnetic field adjustment device can obtain more comprehensive magnetic field information, allowing for finer and more thorough magnetic field adjustments. Since the Hall detection units are capable of sensing changes in the magnetic field in real time, the detection component can provide real-time magnetic field information, enabling the control component to promptly perform calculations and adjustments based on the magnetic field information, thereby maintaining the stability and accuracy of the magnetic field. The installation positions of the Hall detection units are adjustable and flexible and may be freely selected according to the structure and requirements of the particle accelerator to meet the needs of magnetic field monitoring under different conditions. In summary, the detection component utilizing the Hall detection unit(s) provides high-precision and real-time magnetic field information, which offers reliable data support for the control component, enabling the magnetic field adjustment device to more accurately adjust the particle deflection magnetic field and maintaining the stability and accuracy of the particle beam, thereby improving the effectiveness of the arc therapy.

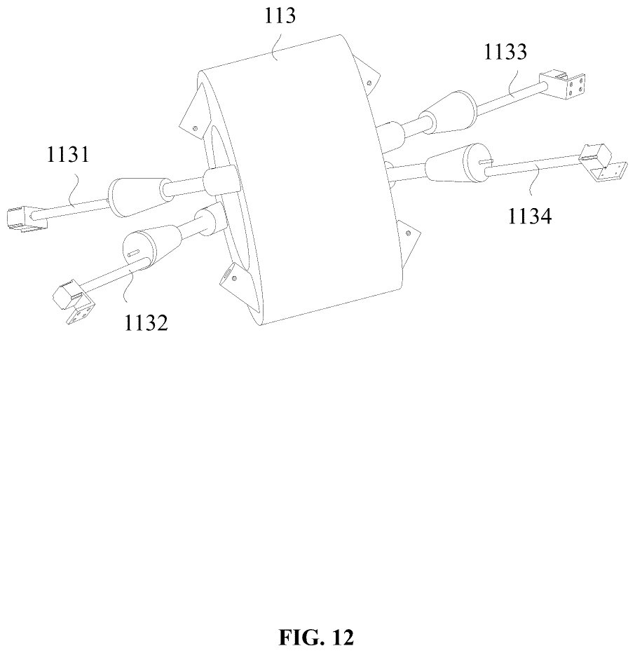

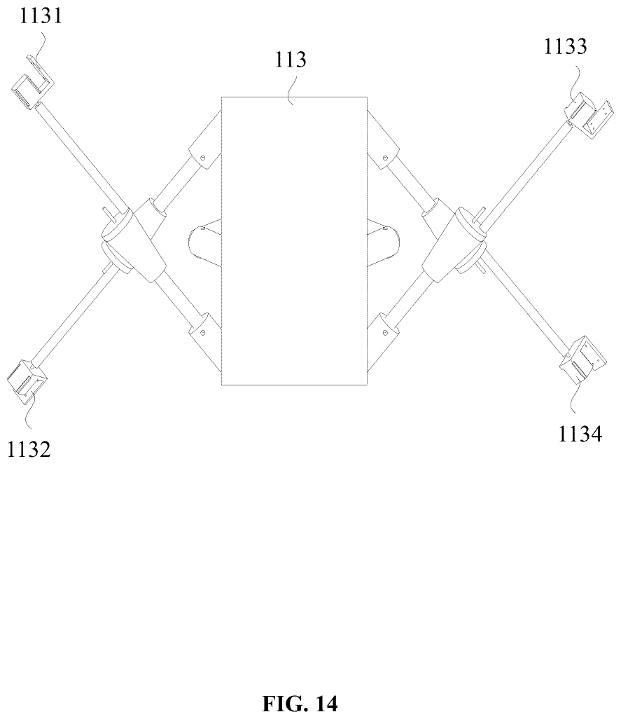

FIG. 12 is a three-dimensional view of a magnetic field providing device and execution components according to some embodiments of the present disclosure. FIG. 13 is a side view of a magnetic field providing device and execution components according to some embodiments of the present disclosure. FIG. 14 is a top view of a magnetic field providing device and execution components according to some embodiments of the present disclosure.

In some embodiments, as shown in FIGS. 12-14, the at least one execution component 1130 includes a first execution component 1131, a second execution component 1132, a third execution component 1133, and a fourth execution component 1132. The first execution component 1131 and the second execution component 1132 are located on a first side of the magnetic field providing device 113, and the third execution component 1133 and the fourth execution component 1134 are located on a second side of the magnetic field providing device 113. The first side and the second side are arranged opposite to each other. A first connecting line connecting a center of the first execution component 1131 and a center of the fourth execution component 1134 intersects with a second connecting line connecting a center of the second execution component 1132 and a center of the third execution component 1133.

In some embodiments, the first execution component 1131 is disposed on the first side of the magnetic field providing device 113, and is configured to adjust a position of a point of action of the first execution component 1131 or a direction of a force applied by the first execution component 1131 based on a corresponding first control instruction generated by the control component 1120, thereby participating in fine-tuning the local position of the magnetic field providing device and ensuring the uniformity of magnetic field distribution.

In some embodiments, the second execution component 1132 is disposed on the first side of the magnetic field providing device 113, and works in concert with the first execution component 1131. By independently adjusting a displacement or an angle of the second execution component 1132, the second execution component 1132 assists in achieving precise attitude control of the magnetic field providing device 113 in a required direction.

In some embodiments, the third execution component 1133 is disposed on the second side (opposite to the first side) of the magnetic field providing device 113 and is configured to operate based on a corresponding first control instruction generated by the control component 1120. The third execution component 1133 balances or compensates for the forces applied by the execution components (e.g., the first actuating component 1131 and the second actuating component 1132) on the first side, thereby improving the stability and symmetry of magnetic field adjustment.

In some embodiments, the fourth execution component 1134 is disposed on the second side of the magnetic field providing device 113, and operates in coordination with the third execution component 1133. The fourth execution component 1134 is arranged such that the connecting line (e.g., the first connecting line) between the first execution component 1131 and the fourth execution component 1134 intersects with the connecting line (e.g., the second connecting line) between the second execution component 1132 and the third execution component 1133, forming a structurally more stable four-point support/drive configuration, which enhances the capability of the execution components to control the spatial attitude of the magnetic field providing device 113.

The Hall detection unit proposed in the embodiments of the present disclosure is a precision magnetic field sensor capable of accurately measuring the magnetic field information of the particle deflection magnetic field, ensuring that the magnetic field adjustment device can acquire high-quality magnetic field data and providing an accurate basis for subsequent adaptive control. The detection component adopts at least one Hall detection unit. When a plurality of Hall detection units are used, magnetic field changes can be simultaneously detected at different locations. By performing detection at multiple locations, the magnetic field adjustment device can obtain more comprehensive magnetic field information, making the adjustment of the particle deflection magnetic field more refined and thorough. As the Hall detection unit is capable of sensing changes in the magnetic field in real time, the detection component can provide real-time magnetic field information, enabling the control component to promptly perform calculations and adjustments based on the magnetic field information, thereby maintaining the stability and accuracy of the magnetic field. The installation positions of the Hall detection units are adjustable and flexible, and may be freely selected based on the structure and requirements of the particle accelerator to meet magnetic field monitoring needs under various conditions. Overall, the detection component using the Hall detection unit(s) provides high-precision and real-time magnetic field information, which offers reliable data support to the control component, enabling the magnetic field adjustment device to more accurately adjust the particle deflection magnetic field and maintaining the stability and accuracy of the particle beam, thereby improving the effectiveness of the arc therapy.

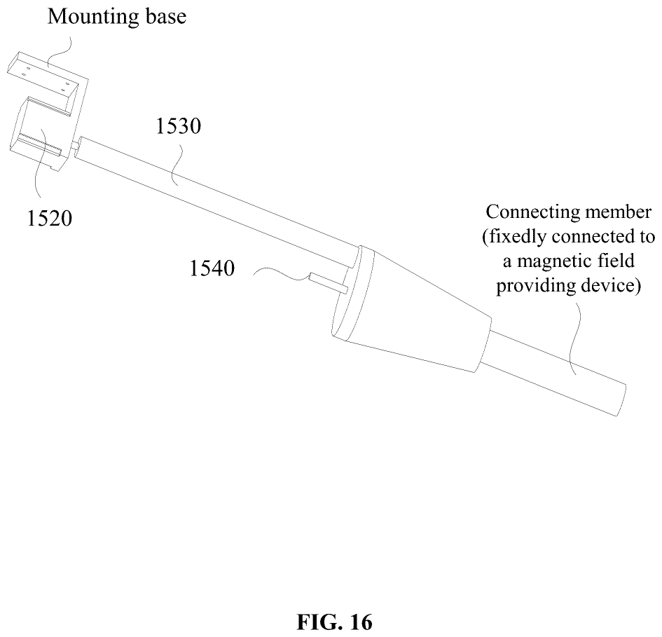

FIG. 15 is a structural block diagram of an execution component according to some embodiments of the present disclosure. FIG. 16 is a schematic diagram of an exemplary structure of an execution component according to some embodiments of the present disclosure.

In some embodiments, as shown in FIGS. 15 and 16, each of the at least one execution component 1130 includes a first drive controller 1510, a first drive mechanism 1520, and a first transmission mechanism 1530. In each of the at least one execution component 1130, the first transmission mechanism 1530 is fixedly connected to the magnetic field providing device 113, and the first drive controller 1510 is configured to drive the first drive mechanism 1520 to move based on a first control instruction corresponding to the execution component 1130, such that the first drive mechanism 1520 drives the first transmission mechanism 1530 to move by a target displacement, to adjust a position and/or an orientation of the magnetic field providing device 113. The control component 1120 is configured to determine the target displacement of the first transmission mechanism 1530 based on motion control information, and generate the first control instruction corresponding to the execution component 1130 to which the first transmission mechanism 1530 belongs based on the target displacement. More descriptions regarding the first control instruction and the motion control information may be found in the related descriptions of FIG. 11.

The first drive controller 1510 is a controller in the execution component 113 and is configured to receive the first control instruction corresponding to the execution component and to control the movement of the first drive mechanism 1520 based on the first control instruction corresponding to the execution component 1130.

The first drive mechanism 1520 refers to a mechanism for executing actual motion, which is configured to perform corresponding movement based on instructions (e.g., the first control instruction) from the first drive controller 1510.

In some embodiments, as shown in FIG. 16, the first drive mechanism 1520 is provided with a housing that is fixedly connected to the treatment gantry 1140 (or a portion of the particle accelerator that remains stationary relative to the treatment gantry) via a mounting base.

The first transmission mechanism 1530 refers to a mechanical structure that converts the power generated by the first drive mechanism 1520 into changes in the position or the orientation of the magnetic field providing device 113. For example, the first transmission mechanism 1530 may include a guide rail slider system, a turntable, or the like.

In some embodiments, the first transmission mechanism 1530 is fixedly connected to the magnetic field providing device 113. As shown in FIG. 16, the first transmission mechanism 1530 is provided with a connecting member, and the first transmission mechanism 1530 is fixedly connected to the magnetic field providing device 113 via the connecting member. The magnetic field providing device 113 may be provided with a connection structure that mates with the connecting member. The first transmission mechanism 1530 is capable of transmitting the movement of the first drive mechanism 1520 to the magnetic field providing device 113, thereby enabling adjustments of the position and/or the orientation of the magnetic field providing device 113 to adjust the particle deflection magnetic field. By adjusting the position and/or the orientation of the magnetic field providing device 113, it can affect the particle deflection magnetic field, thereby changing the energy level, the beam spot size, and the beam spot position of the particle beam to ultimately meet the particle beam condition. The present disclosure does not limit the form of motion of the first transmission mechanism 1530, which may include translation and/or rotation.

The target displacement refers to a preset displacement that the first transmission mechanism 1530 needs to move in order to bring the magnetic field providing device to a preset desired state (i.e., satisfying the particle beam condition).

In some embodiments, the target displacement may be set based on the requirements of magnetic field adjustments, such that the first drive mechanism 203 is adjusted to a target position. During the arc therapy, as the treatment gantry 140 rotates, parameters of the particle beam need to be adjusted according to the treatment plan. Each of the at least one execution component 1130 adjusts the position and/or the orientation of the magnetic field providing device 113 by receiving the target displacement of the first transmission mechanism 1530 corresponding to execution component 1130.

In some embodiments, the target displacement of the first transmission mechanism 1530 corresponding to each preset angular interval may be determined in real time based on the magnetic field information obtained from real-time measurements and the preset magnetic field condition after rotating the treatment gantry.

In some embodiments, the target displacement of the first transmission mechanism 1530 corresponding to each preset angular interval may be obtained by pre-calibration and specified in the treatment plan to guide position adjustments of the magnetic field providing device 112. The pre-calibration can reduce the amount of computation during treatment, reduce the delay caused by computation, and improve the accuracy of motion control and the efficiency of the treatment. Merely by way of example, the target displacement of the first transmission mechanism 1530 corresponding to each preset angular interval is pre-set based on the treatment plan during the rotation of the treatment gantry. For example, the target displacement of each first transmission mechanism 1530 corresponding to the preset angles 0°, 45°, 90°, 135°, and 180° may be pre-calibrated.

In some embodiments, the target displacement may be determined based on a difference between the magnetic field information and the preset magnetic field condition analyzed by the control component 1120. For example, the control component 1120 first determines a required adjustment magnitude of the magnetic field providing device based on required changes in the strength and the direction of the magnetic field, and then converts the required adjustment magnitude into a displacement corresponding to the first transmission mechanism.

In some embodiments, as shown in FIGS. 12-14, the first transmission mechanism 1530 of each of the first execution component 1131, the second execution component 1132, the third execution component 1133, and the fourth execution component 1134 is capable of translating along a preset straight line corresponding to the first transmission mechanism 1530, to move toward or away from the magnetic field providing device. Each of the first transmission mechanisms 1530 may translate its corresponding target displacement along its corresponding preset straight path. The translation is directional, i.e., the first transmission mechanism may move close to or away from the magnetic field providing device 113.

In some embodiments, as shown in FIGS. 12-14, the first transmission mechanisms 1530 of the execution components 1130 provided on opposite sides of the magnetic field providing device 113 (e.g., the first execution component 1131 is provided opposite to the fourth execution component 1134, and the second execution component 1132 is provided opposite to the third execution component 1133) may collaboratively achieve angular control of the magnetic field providing device 113. For example, the first transmission mechanism 1530 of the first execution component 1131 on an upper left side of the magnetic field providing device 113 translates a distance in a direction away from the magnetic field providing device 113 (e.g., a direction back from a connection point between the first transmission mechanism 1530 of the first execution component 1131 and the magnetic field providing device 113), and the first transmission mechanism 1530 of the fourth execution component 1134 on a lower right side of the magnetic field providing device 113 translates in the direction away from the magnetic field providing device 113, then the magnetic field providing device 113 undergoes rotation around an axis of the magnetic field providing device 113.

The magnetic field providing device 113 has three attitude angles. If a plurality of attitude angles of the magnetic field providing device 113 needs to be adjusted, the first transmission mechanism 1530 of the second execution component 1132 on the lower left side of the magnetic field providing device 113 and the first transmission mechanism 1530 of the third execution component 1133 on the upper right side of the magnetic field providing device 113 may perform appropriate translational movements to realize an orientation adjustment of the magnetic field providing device 113. If the magnetic field providing device 113 needs to be translated, the first transmission mechanisms 1530 of the first execution component 1131 and the second execution component 1132 on a left side of the magnetic field providing device 113 may be controlled to translate in the direction away from the magnetic field providing device 113. At the same time, the first transmission mechanisms 1530 of the third execution component 1133 and the fourth execution component 1134 on a right side of the magnetic field providing device 113 may be controlled to translate in a direction close to the magnetic field providing device (e.g., in a direction toward connection points between the first transmission mechanisms 1530 of the third execution component 1133 and the fourth execution component 1134 and the magnetic field providing device 113). Thereby, a position adjustment of the magnetic field providing device 113 is realized under the combined pulling forces on the first side and the second side (e.g., the left side and the right side) of the magnetic field providing device 113.

For example, in the particle accelerator of the arc therapy apparatus 100, the magnetic field adjustment device 140 is used to adjust the energy level of the particle beam. The magnetic field adjustment device 140 includes four execution components 1130, each of the four execution components 1130 receives a corresponding first control instruction.

In each of the plurality of execution components 1130, the first drive controller 1510 receives the first control instruction corresponding to the execution component 1130, controls the movement of the first drive mechanism 1520, and drives, through the first transmission mechanism 1530, the magnetic field providing device 113 to change the position and/or the orientation of the magnetic field providing device 113, thereby realizing the position adjustment and/or the orientation adjustment of the magnetic field providing device 113. For example, if it is required to reduce the energy level of the particle beam from 230 MeV to 229 MeV, the first drive controllers 1510 of the execution components 1130 may move the magnetic field providing device 113 to the required position and/or the required orientation via the first drive mechanisms 1520 and the first transmission mechanisms 1530, such that the energy level of the particle beam is adjusted. As another example, if it is required to reduce the beam spot size of the particle beam from 1.5 cm to 1.2 cm, the first drive controllers 1510 may adjust the position and/or the orientation of the magnetic field providing device 113 via the first drive mechanisms 1520 the first transmission mechanisms 1530, such that the beam spot size of the particle beam is adjusted. As a further example, if it is required to move the beam spot position of the particle beam from a left side to a right side of a subject (e.g., a patient), the first drive controllers 1510 may adjust the magnetic field providing device 113 to the required position and/or the required orientation through the first drive mechanisms 1520 and the first transmission mechanisms 1530, such that the beam spot position of the particle beam is adjusted.

In some embodiments of the present disclosure, each of the at least one execution component is provided with an independent first drive controller and a first drive mechanism, which enables the magnetic field adjustment device to realize precise control of the particle deflection magnetic field. By controlling the movement of the first transmission mechanism and the magnetic field providing device, the position and/or the orientation of the particle deflection magnetic field can be accurately adjusted, thereby enabling precise radiation therapy. Each of the at least one execution component is an independent unit, and movements of the at least one execution component can be adjusted relatively independently, so that the magnetic field adjustment device can flexibly adapt to the needs of different treatment scenarios and different subjects, thereby achieving more personalized and customized radiation therapy. The first drive controller of each of the at least one execution component drives the movement of the first drive mechanism of the execution component based on the first control instruction corresponding to the execution component, and the first drive mechanism drives the first transmission mechanism to achieve real-time adjustments of the magnetic field providing device. This configuration allows the magnetic field adjustment device to quickly respond to actual magnetic field changes and maintain the stability and consistency of particle beam parameters. Through precise control of the position and/or the orientation of the magnetic field providing device, the particle deflection magnetic field generated by the magnetic field adjustment device enables highly controllable manipulation of the particle beam parameters, thereby improving the accuracy and effectiveness of radiation therapy and minimizing damage to healthy tissues to the greatest extent possible.

In some embodiments, as shown in FIG. 15, each of the at least one execution component 1130 further includes a position detection mechanism 1540. In each of the at least one execution component 1130, the position detection mechanism 1540 is configured to: detect and obtain a real-time displacement of the first transmission mechanism 1530 and transmit the real-time displacement to the control component 1120, such that the control component 1120 generates a new first control instruction based on the real-time displacement and the target displacement of the first transmission mechanism 1530, and transmits the new first control instruction to the first drive controller 1510. The first drive controller 1510 is configured to drive the first drive mechanism 1520 based on the new first control instruction, such that the first drive mechanism 1520 drives the first transmission mechanism 1530 to move, to cause the real-time displacement of the first transmission mechanism 1530 to match the target displacement of the first transmission mechanism 1530.

The position detection mechanism 1540 refers to a feedback device in the execution component 1130. The position detection mechanism 1540 is configured to detect the real-time displacement of the first transmission mechanism 1530 and transmit the real-time displacement data to the control component 1120.

In some embodiments, the position detection mechanism 1540 may be mounted on the first transmission mechanism 1530, or near a mechanical part that links with the first transmission mechanism 1530, ensuring accurate acquisition of a motion state of the first transmission mechanism 1530. For example, the position detection mechanism 1540 may be integrated into a slider, a guide rail, a rotating shaft, etc.

The real-time displacement refers to a real-time movement distance of the first transmission mechanism 1530, which is acquired in real-time by the position detection mechanism 1540.

The new first control instruction refers to a control signal used for correcting the movement of the execution component 1130. The control signal is recalculated and generated by the control component 1120 based on a difference between the real-time displacement and the target displacement.

In some embodiments, the control component 1120 may obtain the real-time displacement of the first transmission mechanism 1530 through the position detection mechanism 1540, compare the real-time displacement with the target displacement, determine the difference, and generate the new first control instruction using a control algorithm based on the difference. Then the control component 1120 sends the new first control instruction to the first drive controller 1510 to achieve closed-loop feedback control, ensuring that the magnetic field providing device 113 is positioned accurately.

For each of the at least one execution component 1130, through continuous motion feedback and adjustment, the first transmission mechanism 1530 of the execution component 1130 achieves precise adjustment of the position and/or the orientation of the magnetic field providing device 113, thereby enabling precise adjustment of the particle deflection magnetic field and accurate tuning of the energy level of the particle beam. Other parameters such as the beam spot size and the beam spot position of the particle beam may be adjusted in a similar manner, which is not repeated here.

In some embodiments of the present disclosure, based on the target displacement and the real-time displacement detected by the position detection mechanism 1540, the new first control instruction for each of the at least one execution component 1130 is generated. The new first control instruction is sent to the first drive controller 1510 to drive the first drive mechanism 1520 to move, thereby enabling precise adjustment and control of the magnetic field providing device 113, and ensuring stability and accuracy of the particle beam parameters. The position detection mechanism 1540 can detect the real-time displacement of the first transmission mechanism 1530 in real time, allowing the control component 1120 to generate the first control instruction based on the real-time displacement and the target displacement, thus achieving real-time adjustment and control of the magnetic field providing device 113. The control component 1120 can automatically generate the new first control instruction corresponding to each execution component 1130, offering a high level of automation. By precisely controlling the position and/or the orientation of the magnetic field providing device 113, the magnetic field adjustment device 140 enables a high degree of controllability over the particle beam parameters, thereby enhancing the precision and effectiveness of radiation therapy and minimizing damage to healthy tissues.

In some embodiments, the first drive mechanism 1520 includes a motor; and/or the first transmission mechanism 1530 includes a mechanical shaft; and/or the position detection mechanism 1540 includes a potentiometer.

In some embodiments, the first drive mechanism 1520 includes an electric motor, which is capable of converting electrical energy into mechanical energy. For example, the first drive mechanism 1520 adopts a direct current (DC) motor.

The mechanical shaft refers to a mechanical component that enables a movement of an object by adjusting a position or an orientation of the mechanical shaft. For example, the first transmission mechanism 1530 may adopt a rotary shaft or a linear shaft as the mechanical shaft.

The potentiometer refers to a sensor capable of measuring position or displacement. The potentiometer determines changes in position based on variations in measured voltage. For example, when the first transmission mechanism 1530 moves, the potentiometer may detect a positional change (i.e., a displacement) and send a signal corresponding to the positional change to the control component 1120.

Merely by way of example, when the magnetic field providing device 113 adopts a magnet coil, the mechanical shaft is fixed to a support structure of the magnet coil and may be configured to adjust a position and/or an orientation of the magnetic coil. The motor is driven via a motor controller and controls the motion of the mechanical shaft. The potentiometer is configured to monitor the real-time displacement of the mechanical shaft, thereby enabling real-time monitoring and control of the position and/or the orientation of the magnetic coil.

In some embodiments of the present disclosure, by using the motor as the first drive mechanism, it enables accurate and controllable power output, allowing precise and flexible adjustment of the magnetic field providing device, thereby meeting complex magnetic field regulation requirements. By using the mechanical shaft as the first transmission mechanism, it effectively converts the motor's rotational motion into a required linear adjustment or a required rotational adjustment, ensuring the stability and reliability of the adjustments. By using the potentiometer as the position detection mechanism, it allows for high-precision real-time monitoring of the positional change of the first transmission mechanism, ensuring quick responses to correct any deviations, thus maintaining the magnetic field providing device in an optimal working state. Overall, these design improves the precision, responsiveness, and stability of the arc therapy apparatus, contributing to more efficient and accurate radiation therapy outcomes.

During the rotation of the treatment gantry, a Hall probe senses a strength of the particle deflection magnetic field in the particle accelerator and feeds the magnetic field information back to the control component 1120. The control component 1120 determines motion control information, such as displacement data (i.e., the target displacement) that each mechanical shaft needs to be adjusted, based on the magnetic field information and the preset magnetic field condition, and commands the motor controller to drive the motor, which drives the magnet coil through the mechanical shaft. The potentiometer monitors a movement distance of the mechanical shaft in real time and feeds the movement distance back to the control component 1120. Through multiple rounds of motion and feedback, the motor drives the mechanical shaft to a preset position, thereby positioning the magnetic coil at its corresponding target position and/or target orientation (i.e., target direction).

As the treatment gantry 140 rotates from a preset angular interval to a next preset angular interval, a change of the gantry angle may cause a change in the particle deflection magnetic field, and the Hall probe continuously monitors the strength of the particle deflection magnetic field in the particle accelerator, and continually drives the motion of the entire execution component 1130 until the particle deflection magnetic field in the cavity 112 of the particle accelerator 110 reaches an acceptable range (i.e., the preset magnetic field condition).

In some embodiments of the present disclosure, the strength of the particle deflection magnetic field in the particle accelerator is sensed by the Hall probe and the position of the magnet coil is adjusted in real time by the execution component, thereby achieving precise adjustment of the particle deflection magnetic field, and improving the accuracy and effectiveness of radiation therapy. The position detection mechanism uses the potentiometer to monitor the displacement of the mechanical shaft in real time, allowing the control component to track the position of the mechanical shaft and make timely adjustments, ensuring that the particle deflection magnetic field is adjusted accurately in real time. The control component automatically updates the first control instruction based on the positional feedback from the first transmission mechanism and drives the motor automatically via the motor controller, thereby achieving automated adjustment of the magnetic coil, making the adjustment of the particle deflection magnetic field more intelligent and efficient. By precisely controlling the position and/or the orientation of the magnetic field providing device, the adaptive coil system enables high-level controllability of the particle beam parameters, contributing to improved accuracy and therapeutic outcomes in radiation therapy while minimizing damage to healthy tissues.

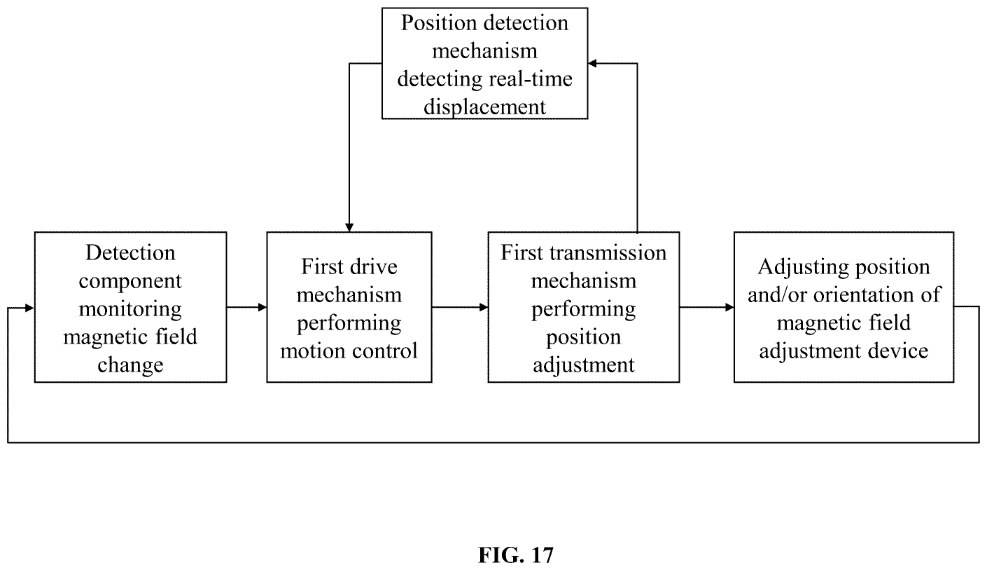

FIG. 17 is a control flowchart of a magnetic field adjustment device according to some embodiments of the present disclosure.

In some embodiments, as shown in FIG. 17, the detection component 1110 first monitors a magnetic field change and transmits the magnetic field change to the first drive mechanism 1520 for motion control, and then the first drive mechanism 1520 drives the first transmission mechanism 1530 to adjust a position of the magnetic field providing device 113, thereby changing the position and orientation of the magnetic field providing device 113. At the same time, the position detection mechanism 1540 monitors a displacement of the first transmission mechanism 1530 in real time and feeds the displacement back to the control component 1120 to ensure that the adjustment accurately matches a target requirement (e.g., a target displacement), forming a closed loop for high-precision control.

As shown in FIG. 17, for each execution component, the control component 1120 may perform two types of closed-loop control.

The first type of closed-loop control (also referred to as large closed-loop control): the detection component 1110 detects a change in the magnetic field; the control component 1120 determines (or retrieves pre-calibrated) motion control information and generates a first control instruction; the first drive controller 1510 drives the first drive mechanism 1520 to move; the first drive mechanism 1520 drives the first transmission mechanism 1530 to move, to adjust a position of the first transmission mechanism 1530; and a position and/or an orientation of the magnetic field providing device 113 is adjusted. The first drive controller 1510 is configured to drive the first drive mechanism 1520 to move based on the first control instruction corresponding to the execution component 1130, so as to cause the first drive mechanism 1520 to drive the first transmission mechanism 1530 to move by the target displacement, thereby adjusting the position and/or the orientation of the magnetic field providing device 113.

The second type of closed-loop control (also referred to as small closed-loop control): the position detection mechanism 1540 detects a real-time displacement; the control component 1120 generates a new first control command based on the real-time displacement and the target displacement; the first drive controller 1510 drives the first drive mechanism 1520 to move; the first drive mechanism 1520 drives the first transmission mechanism 1530 to move, to adjust the position of the first transmission mechanism 1530; and the position and/or the orientation of the magnetic field providing device 113 is adjusted. The position detection mechanism 1540 is configured to detect the real-time displacement of the first transmission mechanism 1530 and send the real-time displacement to the control component 1120, so as to cause the control component 1120 to generate the new first control instruction based on the real-time displacement of the first transmission mechanism 1530 and the target displacement. The first drive controller 1510 is configured to drive the first drive mechanism 1520 to move based on the new first control instruction and drive the first transmission mechanism 1530 to move through the first drive mechanism 1520, thereby ensuring that the real-time displacement of the first transmission mechanism 1530 matches with the target displacement.

The real-time displacement of the first transmission mechanism matching with the target displacement means that the real-time displacement and the target displacement are exactly the same, or an absolute value of a difference between the real-time displacement and the target displacement is less than a preset value, or a ratio of the absolute value to the target displacement is less than a preset ratio.

By way of example, the magnetic field adjustment device 140 utilizes an adaptive coil system including two Hall probes, four execution components, and one control component. The Hall probes are disposed inside the particle accelerator to generate a voltage proportional to the measured magnetic field for position feedback of a magnet coil. Each of the four execution components includes a mechanical shaft, a motor, a motor controller, and a potentiometer. Each of the four mechanical shafts is fixed to a support structure of the magnet coil and may effectively adjust a relative position and an orientation of the magnet coil. The potentiometer is configured to determine the position of the mechanical shaft to realize the monitoring and control of the adaptive coil system.

A control logic of the adaptive coil system is shown in FIG. 17. During the rotation of the treatment gantry, the Hall probes monitor an anomaly in the strength of the magnetic field of the particle accelerator (an anomaly judgment condition may be predefined, e.g., a mismatch between the measured magnetic flux density and a predefined value at a current position) and feeds the anomaly back to the control system. Based on current magnetic field information and the preset magnetic field condition, the control component determines an adjustment (e.g., the target displacement) that needs to be made for each mechanical shaft and commands the motor controllers to start driving the motors. The motors then move the mechanical shafts along predefined linear paths, either toward or away from the magnetic field providing device.

The potentiometers monitor the movement distances (i.e., real-time displacements) of the mechanical shafts in real time and feed back to the motor controllers. After multiple rounds of movement and feedback, the motors drive each of the mechanical shafts to reach a preset position, at which time the movement distance of each of the mechanical shafts reaches the corresponding target displacement. Accordingly, the magnet coil reaches a preset position. The Hall probes continues to monitor the strength of the magnetic field in the particle accelerator and continuously drive the entire adaptive coil system until the particle deflection magnetic field reaches an acceptable range.

Embodiments of the present disclosure further provide a non-transitory computer-readable storage medium. Specific embodiments of the storage medium are consistent with the embodiments described in the aforementioned method and achieve the same technical effects, which is not repeated.

The non-transitory computer-readable storage medium stores a computer program, which, when executed by at least one processor, implements the operations of any of the aforementioned methods or the functions of any of the aforementioned electronic devices.

The non-transitory computer-readable storage medium may be a non-transitory computer-readable signaling medium or a non-transitory computer-readable storage medium. In embodiments of the present disclosure, the non-transitory computer-readable storage medium may be any tangible medium containing or storing a program, which may be used by, or in conjunction with, an instruction-executing system, apparatus, or device. The non-transitory computer-readable storage medium may include, but is not limited to, electrical, magnetic, optical, electromagnetic, infrared, or semiconductor systems, apparatuses, or devices, or any suitable combination thereof. More specific examples of the non-transitory computer-readable storage medium include: electrical connections with one or more wires, portable disks, hard disks, random-access memory (RAM), read-only memory (ROM), erasable programmable read-only memory (EPROM or flash memory), optical fibers, portable compact disk read-only memory (CD-ROM), optical storage devices, magnetic storage devices, or any suitable combination thereof.

The non-transitory computer-readable storage media may include a data signal propagated in a baseband or as part of a carrier carrying readable program code. The propagated data signal may take a variety of forms, including, but not limited to, an electromagnetic signal, an optical signal, or any suitable combination of the foregoing. The non-transitory computer-readable storage medium may also be any non-transitory computer-readable medium capable of transmitting, propagating, or transferring a program for use by or in connection with an instruction execution system, an apparatus, or a device. The program code contained on the non-transitory computer-readable storage medium may be transmitted using any suitable medium including, but not limited to, a wireless medium, a wired medium, a fiber optic cable, radio frequency (RF), etc., or any suitable combination of the foregoing. The program code for performing the operations of the present disclosure may be written in any combination of one or more programming languages, including but not limited to Java, C++, Python, C#, JavaScript, PHP, Ruby, Swift, Go, Kotlin, etc. The program code may execute entirely on a user's computing device, partly on the user's device, as a standalone software package, partly on the user's device and partly on a remote computing device, or entirely on a remote computing device or a server. In scenarios involving a remote computing device, the remote computing device may connect to the user's device via any type of network, including a local area network (LAN) or a wide area network (WAN), or may connect to an external computing device (e.g., via an internet service provider over the Internet).

Some embodiments of the present disclosure further provide a computer program product. Specific embodiments of the computer program product are consistent with the embodiments described in the aforementioned method and achieve the same technical effects, and the details are not repeated.

The computer program product includes a computer program, which, when executed by at least one processor, implements the operations of any of the aforementioned methods or the functions of any of the aforementioned electronic devices.

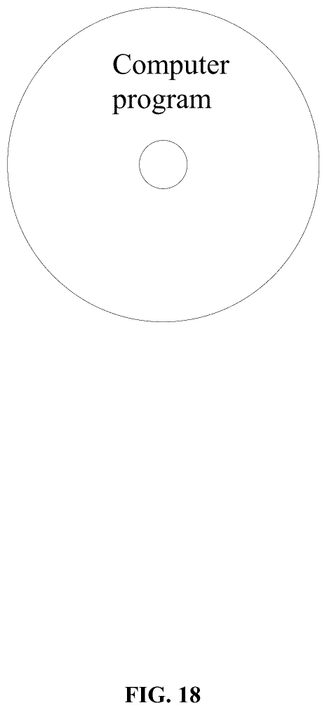

FIG. 18 is a schematic diagram of an exemplary structure of a computer program product according to some embodiments of the present disclosure. In some embodiments, as shown in FIG. 18, the computer program product is configured to implement the operations of any of the aforementioned methods or the functions of any of the aforementioned operational devices. The computer program product may adopt a portable compact disc read-only memory (CD-ROM) containing program code and may run on a terminal device such as a computer. However, the computer program product of the present disclosure is not limited thereto and may utilize any combination of one or more non-transitory computer-readable media.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

It should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This way of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameter set forth in the written description and attached claims are approximations that may vary depending upon the desired properties sought to be obtained by a particular embodiment. In some embodiments, the numerical parameter should be construed in light of the count of reported significant digits and by applying ordinary rounding techniques. Notwithstanding that the numerical ranges and parameter setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrating of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A magnetic field adjustment device, applied to a particle accelerator mounted on a treatment gantry of an arc therapy apparatus, the particle accelerator rotating along with the treatment gantry during rotation of the treatment gantry, wherein
the particle accelerator comprises a magnetic field providing device configured to provide a particle deflection magnetic field;
the magnetic field adjustment device is configured to:
adjust the particle deflection magnetic field based on a change of a gantry angle of the treatment gantry, such that a particle beam generated by a particle radiation source satisfies a preset particle beam condition under the particle deflection magnetic field; wherein
the preset particle beam condition indicates a preset range of each of one or more of an energy level, a beam spot size, and a beam spot position of the particle beam.

2. The magnetic field adjustment device of claim 1, wherein the magnetic field adjustment device comprises a detection component, a control component, and at least one execution component;
the detection component is mounted inside the particle accelerator and is configured to detect magnetic field information of the particle deflection magnetic field;
the control component is configured to determine motion control information of the magnetic field providing device based on the magnetic field information and a preset magnetic field condition, and generate a first control instruction corresponding to each of the at least one execution component based on the motion control information, wherein the preset magnetic field condition is determined based on the preset particle beam condition; and
each of the at least one execution component is connected to the control component and the magnetic field providing device, and is configured to adjust a position and/or an orientation of the magnetic field providing device based on the first control instruction corresponding to the execution component.

3. The magnetic field adjustment device of claim 2, wherein
the detection component includes at least one Hall detection unit configured to detect a magnetic field at at least one location of the particle deflection magnetic field to obtain the magnetic field information; and/or
the at least one execution component includes a first execution component, a second execution component, a third execution component, and a fourth execution component, wherein
the first execution component and the second execution component are located on a first side of the magnetic field providing device, the third execution component and the fourth execution component are located on a second side of the magnetic field providing device, and the first side and the second side are arranged opposite to each other; and
a first connecting line connecting a center of the first execution component and a center of the fourth execution component intersects with a second connecting line connecting a center of the second execution component and a center of the third execution component.

4. The magnetic field adjustment device of claim 2, wherein each of the at least one execution component includes a first drive controller, a first drive mechanism, and a first transmission mechanism;
in each of the at least one execution component, the first transmission mechanism is fixedly connected to the magnetic field providing device, and the first drive controller is configured to drive the first drive mechanism to move based on the first control instruction corresponding to the execution component, such that the first drive mechanism drives the first transmission mechanism to move by a target displacement, to adjust the position and/or the orientation of the magnetic field providing device; and
the control component is configured to determine the target displacement of the first transmission mechanism based on the motion control information, and generate the first control instruction corresponding to the execution component to which the first transmission mechanism belongs based on the target displacement.

5. The magnetic field adjustment device of claim 4, wherein each of the at least one execution component further includes a position detection mechanism, and in each of the at least one execution component,
the position detection mechanism is configured to detect a real-time displacement of the first transmission mechanism and transmit the real-time displacement to the control component, such that the control component generates a new first control instruction based on the real-time displacement and the target displacement of the first transmission mechanism, and transmits the new first control instruction to the first drive controller; and
the first drive controller is configured to drive the first drive mechanism to move based on the new first control instruction, such that the first drive mechanism drives the first transmission mechanism to move, to cause the real-time displacement of the first transmission mechanism to match the target displacement of the first transmission mechanism.

6. The magnetic field adjustment device of claim 5, wherein the first drive mechanism includes a motor; and/or
the first transmission mechanism includes a mechanical shaft; and/or
the position detection mechanism includes a potentiometer.

7. An arc therapy apparatus, comprising:
a particle accelerator configured to generate and adjust a particle beam;
a beam delivery system configured to deliver the particle beam;
a subject positioning system configured to position a subject; and
a treatment gantry configured to drive the particle accelerator and the beam delivery system to rotate around an isocenter to perform arc therapy.

8. The arc therapy apparatus of claim 7, wherein the particle accelerator includes:
a particle radiation source configured to generate the particle beam, wherein particles of the particle beam are protons or heavy ions;
a cavity configured to accelerate the particle beam within the cavity; and
a magnetic field providing device configured to provide a particle deflection magnetic field for the cavity.

9. The arc therapy apparatus of claim 8, wherein the particle accelerator further includes a beam spot size adjustment device configured to adjust a beam spot size of the particle beam.

10. The arc therapy apparatus of claim 9, wherein the beam spot size adjustment device includes at least one adjustment component, each of the at least one adjustment component includes a second drive controller, a second drive mechanism, a second transmission mechanism, and a blocking mechanism; wherein
in each of the at least one adjustment component, the second transmission mechanism is fixedly connected to the blocking mechanism, and the second drive controller is configured to drive the second drive mechanism to move based on a second control instruction corresponding to the adjustment component, such that the second drive mechanism drives the second transmission mechanism to move, to adjust a blocking state of the blocking mechanism.

11. The arc therapy apparatus of claim 10, wherein
the second drive mechanism includes a motor; and/or
the second transmission mechanism includes a lead screw; and/or
the blocking mechanism includes a plurality of shielding plates arranged in pairs, each pair of the shielding plates being symmetrically arranged along a central line of the blocking mechanism.

12. The arc therapy apparatus of claim 8, wherein the particle accelerator further includes
a radio frequency (RF) device configured to provide an accelerating electric field for the cavity of the particle accelerator; and/or
a vacuum device configured to provide a vacuum environment for the cavity; and/or
a liquid cooling device configured to cool the cavity through a cooling liquid.

13. The arc therapy apparatus of claim 7, wherein
the beam delivery system includes a treatment head, and the treatment head includes an active beam scanning treatment head and/or a passive scattering treatment head; and/or
the subject positioning system includes a treatment couch and an imaging system.

14. The arc therapy apparatus of claim 7, further comprising:
a safety interlock system configured to perform safety monitoring on the particle accelerator, the beam delivery system, the subject positioning system, and the treatment gantry; and/or
a treatment planning system configured to generate a treatment plan based on preoperative medical imaging data of the subject; and/or
a control software system configured to verify the treatment plan and record treatment process data.

15. A method for operating an arc therapy apparatus, comprising:
acquiring a treatment plan, the treatment plan including a plurality of preset angular intervals and a preset dose of a particle beam corresponding to each of the preset angular intervals; and
rotating a treatment gantry of the arc therapy apparatus to each of the preset angular intervals, respectively, to complete an irradiation process of the particle beam corresponding to each of the preset angular intervals.

16. The method of claim 15, wherein before rotating the treatment gantry, the method further comprises:
adjusting a treatment couch to a preset irradiation position;
acquiring first intraoperative medical imaging data of a subject through an imaging system;
performing image registration between the first intraoperative medical imaging data of the subject and the treatment plan to obtain treatment couch adjustment information; and
adjusting a position and an angle of the treatment couch based on the treatment couch adjustment information.

17. The method of claim 16, wherein after adjusting the position and the angle of the treatment couch, the method further comprises:
acquiring second intraoperative medical imaging data of the subject through the imaging system;
determining whether image registration between the second intraoperative medical imaging data and the treatment plan is completed;
in response to determining that the image registration between the second intraoperative medical imaging data and the treatment plan is completed, rotating the treatment gantry; and
in response to determining that the image registration between the second intraoperative medical imaging data and the treatment plan is not completed, reacquiring the treatment couch adjustment information to readjust the treatment couch.

18. The method of claim 15, wherein the preset angular intervals are represented by discrete preset angles, and one or more of the following operations are performed during arc therapy:
S1: rotating the treatment gantry to a first preset angle;
S2: extending a treatment head to initiate irradiation of the particle beam;
S3: at the preset angle, monitoring an irradiation dose of the particle beam, and stopping the irradiation when the irradiation dose reaches the preset dose corresponding to the preset angle;
S4: retracting the treatment head;
S5: determining whether a next preset angle exists; in response to determining that the next preset angle exists, proceeding to S6; in response to determining that the next preset angle does not exist, proceeding to S7;
S6: rotating the treatment gantry to the next preset angle and proceeding to S2-S5; and
S7: ending the arc therapy.

19. The method of claim 15, wherein the preset angular intervals are represented by angular ranges, and one or more of the following operations are performed during arc therapy:
R1: rotating the treatment gantry to a first preset angular range;
R2: extending a treatment head to initiate irradiation of the particle beam;
R3: within the preset angular range, monitoring an irradiation dose of the particle beam, and when the irradiation dose reaches the preset dose corresponding to the preset angular range, determining whether a next preset angular range exists; in response to determining that the next preset angular range exists, proceeding to R4; in response to determining that the next preset angular range does not exist, proceeding to R5;
R4: rotating the treatment gantry to the next preset angular range and proceeding to R3; and
R5: stopping the irradiation of the particle beam, retracting the treatment head, and ending the arc therapy.

20. The method of claim 19, wherein an extension position of the treatment head does not exceed a preset position, the preset position being determined based on a preset collision constraint condition.
